(19) United States
(12) Patent Application Publication
(10) Pub. No.: US 2002/0115065 A1
(43) Pub. Date: Aug. 22, 2002

(54) Differentially expressed epitopes and uses thereof

(76) Inventors: Ton Logtenberg, Werkhoven (NL); Lucia Cilenti, Rome (IT); Andries Christiaan Bloem, Driebergen (NL); Renate Marie Louise Zwijsen, Utrecht (NL)

Correspondence Address: TRASK BRITT, P.O. BOX 2550, SALT LAKE CITY, UT, 84110, US

(21) Appl. No.: 09/940,386

(22) Filed: Aug. 27, 2001

Related U.S. Application Data
(60) Provisional application No. 60/228,429, filed on Aug. 28, 2000.

(51) Int. Cl. C12Q 1/70; C12Q 1/68; G01N 33/53; G01N 33/567; G01N 33/554; G01N 33/569

(52) U.S. Cl. 435/5; 435/7.1; 435/7.21; 435/7.32; 435/6

(57) ABSTRACT

Provided are, among other things, novel epitopes, methods for finding these epitopes and binding molecules capable of binding to said novel epitopes. In one aspect, the invention provides a binding molecule capable of specifically binding to a preferably, post-translationally modified, disease associated molecular marker, associated with diseased cells, whereas the preferably post-translationally modified disease associated molecular marker is not associated with non-diseased cells. In a preferred aspect of the invention, the binding molecule recognizes an epitope present in a subset of CD46 proteins. The binding molecule is capable of distinguishing between CD46 proteins belonging to the subset and CD46 proteins not belonging to the subset. The binding molecule is preferably an antibody. Medicaments and uses of binding molecules are provided.

### CROSS REFERENCE TO RELATED APPLICATION

Under the provisions of 35 U.S.C. § 119(e), priority is claimed from U.S. Provisional Patent Application Ser. No. 60/228,429, filed Aug. 28, 2001, the entirety of which is hereby incorporated herein by this reference.

### TECHNICAL FIELD

The invention relates to the field of medicine. The invention further relates to disease-associated molecular markers and methods of obtaining and using these markers. The invention also relates to the diagnostic and/or medical use of binding molecules capable of recognizing and/or binding the disease-associated molecular markers.

### BACKGROUND

B-lymphocytes can produce antibodies in response to exposure to biological substances like bacteria, viruses and their toxic products. Antibodies are generally epitope specific and bind strongly to the biological substances carrying these epitopes. The hybridoma technique (Köhler and Milstein 1975) makes use of the ability of the B cells to produce monoclonal antibodies to specific antigens and to subsequently isolate and produce monoclonal antibodies by fusing B cells from mice exposed to the antigen of interest to immortalized murine plasma cells. This technology resulted in the realization that monoclonal antibodies produced by hybridomas could be used in research, diagnostics and therapies to treat different kinds of diseases like cancer and auto-immune related disorders.

Because antibodies that were produced in mouse hybridomas induced dramatic immune responses in humans, it became clear that the antibodies that were required for successful treatments of diseases in human needed to fit the human requirements to lower these immune responses. For this, murine antibodies were first engineered by replacing the murine constant domains with human constant regions. Subsequently, domains between the variable domains, which specify the antigen binding, were replaced by their human counterparts. The final stage in this humanization process is the production of fully human antibodies.

To date, many different diseases are being treated with either humanized or fully human antibodies. Nevertheless, many disorders are not being treated since no specific epitopes are found, that are expressed on cells or substances that need to be removed by the immune system through the interaction with antibodies or other binding molecules capable of binding the specific epitope.

Much interest is currently directed toward the use of binding molecules in the treatment of human disease. One application is the use of binding molecules to facilitate removal of undesired cells from a body. In this application, the binding molecule must have sufficient specificity for the cells to be removed in order not to result in undesired side effects. Typically, but not necessarily, such binding molecules comprise antibodies. An antibody is capable of binding to an epitope expressed by an undesired cell and thereby mark the cell for removal from the body. This can be done in several ways for instance mediated by the immune system or the complement system or a combination thereof. Removal can also be achieved in (combination with) other ways.

Antibodies made in vivo can be capable of binding strongly to the invading microorganisms or their products that elicited their production and aid in their elimination. As was mentioned above, the activity of the immune system of producing antibodies in response to an invading microorganism has been exploited in the production of monoclonal antibodies, a technology developed by Köhler and Milstein (1975). In the old definition, monoclonal antibodies are all those immunoglobulin molecules that are produced by the progeny of a single B lymphocyte. Conventionally, monoclonal antibodies are obtained by immunizing a mouse with an antigen and fusing the spleen or lymph node B-lymphocytes with an immortalized murine plasma cell line. The ensuing hybrid cell lines, or so-called hybridomas, bear the characteristics of both parental cell types: they are immortal and produce a single species of monoclonal antibody specific for the antigen used to immunize the mouse. The advantage of monoclonal antibodies is that they represent a homogeneous population of immunoglobulin molecules with a pre-defined binding specificity. Over the years, monoclonal antibodies have proven invaluable tools in research and diagnostics.

Soon after the invention of the hybridoma technology, the enormous potential of monoclonal antibodies in human therapy was realized. Because of their high binding specificity, antibodies were hypothesized, among others, to be capable of binding to viruses and bacteria and their toxic products facilitating their elimination. In other applications, monoclonal antibodies were envisaged to specifically bind to tumor cells to promote their eradication or to bind to soluble molecules produced by cells of the immune system to neutralize their activity in harmful chronic inflammatory conditions and/or in autoimmune disease. Indeed, monoclonal antibodies have been described as magic bullets that could be used in the treatment of a wide variety of human diseases (Bodey et al. 2000).

Numerous clinical studies with monoclonal antibodies of non-human (usually murine) origin showed that they performed poorly as a result of their immunogenicity. Upon injection of murine monoclonal antibodies in humans, the human immune system recognizes the murine monoclonal antibodies as foreign proteins resulting in the induction of an immune response against the murine protein (Miller et al. 1983; Shawler et al. 1985). In addition, murine monoclonal antibodies have poor pharmacokinetic properties in humans (Riechmann et al. 1988) and are inefficient in recruiting effector functions of cells of the immune system (Hakimi et al. 1991; Stephens et al. 1995). These issues have spurred the development of alternative strategies to obtain more human monoclonal antibodies for therapy (reviewed in Vaughan et al. 1998).

In one approach of creating more human monoclonal antibodies, the immunoglobulin variable regions of the murine monoclonal antibodies are genetically fused to human immunoglobulin constant regions (FIG. 1). The resulting chimeric monoclonal antibody still contains >30% murine amino acid sequences. Clinical application in humans of chimeric monoclonal antibodies has shown that these proteins retain their immunogenicity in the majority of cases (Khazaeli et al. 1989; Elliot et al. 1994). In another approach, only immunoglobulin variable region sequences relevant for monoclonal antibody specificity are of murine origin; the constant regions of the immunoglobulin molecule as well as the framework regions of the variable region are of human origin (FIG. 1). Clinical application of these ‘humanized’ monoclonal antibodies indicates that these molecules are generally more effective and have no or little intrinsic toxicity or immunogenicity (Jones et al. 1986). However, reconstructing the original affinity and specificity in a humanized version of a murine monoclonal antibody is a time-consuming process and may render the monoclonal antibody not enough human to completely prevent anti-antibody responses (Foote et al. 1992).

These considerations with chimeric and humanized monoclonal antibodies and the recent clinical success of engineered monoclonal antibodies spurred the development of efficient methods for the isolation and production of fully human monoclonal antibodies, the most desirable monoclonal antibody format for clinical application. The conventional methods to obtain murine and humanized monoclonal antibodies and two novel methods for obtaining monoclonal antibodies with complete human sequences are displayed in FIG. 1.

One method of obtaining human monoclonal antibodies employs transgenic mice harboring human immunoglobulin loci in combination with conventional hybridoma technology (Bruggeman and Neuberger 1996; Mendez et al. 1997). In these mice, large portions of human immunoglobulin heavy and light chain loci have been inserted in the mouse germ line while the endogenous murine immunoglobulin loci have been silenced by gene knockout. Immunization of these transgenic mice with an antigen results in the production of human antibodies specific for the antigen. Human monoclonal antibody-producing cell lines can be obtained from these mice by fusing the spleen cells of immunized mice with plasma cell lines in vitro to obtain immortalized monoclonal antibody-secreting hybridomas. Importantly, production of human monoclonal antibodies in transgenic mice depends on immunization procedures and is governed by constraints of the murine immune response. As a consequence, it is difficult if not impossible to obtain antibodies against the mouse's own antigens (auto-antigens), to xenoantigens that have a high degree of homology to murine auto-antigens or to antigens that have poor immunogenic properties such as polysaccharides. These notions have spurred the development of molecular approaches that obviate the need for immunization and cell ‘immortalization’ to obtain human monoclonal antibodies with desired specificities. These strategies are based on immortalization of the immunoglobulin genes encoding the monoclonal antibodies rather than the cell lines producing them.

Another method to obtain fully human monoclonal antibodies with desirable binding properties employs phage display libraries. This is an in vitro, recombinant DNA-based, approach that mimics key features of the humoral immune response (Burton et al. 1994). For the construction of phage display libraries, collections of human monoclonal antibody heavy and light chain variable region genes are expressed on the surface of bacteriophage particles, either in single chain Fv (scFv) or in Fab format. Large libraries of antibody fragment-expressing phages typically contain >10⁹ antibody specificities and may be assembled from the immunoglobulin V regions expressed in the B lymphocytes of immunized or non-immunized individuals. Alternatively, phage display libraries may be constructed from immunoglobulin variable regions that have been partially assembled in vitro to introduce additional antibody diversity in the library (semi-synthetic libraries). For example, in vitro assembled variable regions contain stretches of synthetically produced, randomized or partially randomized DNA in those regions of the molecules that are important for antibody specificity.

Recombinant phages expressing antibody fragments of desirable specificities may be selected from a library by one of several methods. Target antigens are immobilized on a solid phase and subsequently exposed to a phage library to allow binding of phages expressing antibody fragments specific for the solid phase-bound antigen. Non-bound phages are removed by washing and bound phages eluted from the solid phase for infection of Escherichia coli (E. coli) bacteria and subsequent propagation. Multiple rounds of selection and propagation are usually required to sufficiently enrich for phages binding specifically to the target antigen. Phages may also be selected for binding to complex antigens such as complex mixtures of proteins or whole cells. Selection of antibodies on whole cells has the advantage that target antigens are presented in their native configuration, unperturbed by conformational changes that are introduced by immobilizing an antigen to a solid phase. The constraints imposed by the natural immune response and the influence of the immunogenicity of the target antigen do not permit the isolation of monoclonal antibodies against any antigen by conventional hybridoma technology. In phage approaches, these factors do not play a role, allowing the isolation of monoclonal antibodies directed against ‘difficult’ antigens such as auto-antigens, carbohydrates and toxic antigens.

In one particular selection procedure (depicted in FIG. 2), phage display libraries are used in combination with flow cytometry and cell sorting to isolate antibody fragments against molecules expressed on the plasma membrane of subpopulations of eukaryotic cells present in a heterogeneous mixture (U.S. Pat. No. 6,265,150; De Kruif et al. 1995a). These published methods do not describe the processes of the invention described herein.

In the art, a heterogeneous mixture of cells is incubated with the phage library allowing phages to bind to the different cell types. Subsequently, the cells are stained with fluorochrome-labeled monoclonal antibodies to permit identification of the subpopulation of target cells by immunofluorescence analysis and flow cytometry. Target cells and attached phages are collected by flow cytometry and the attached phages are eluted and propagated. This method is rapid, independent of the immunogenicity of the target antigen and yields antibody fragments against molecules in their native configuration. Specific antibodies against very small populations of cells in a heterogeneous mixture can be obtained (De Kruif et al. 1995a and 1996).

For production of intact human monoclonal antibodies, scFv with desirable specificities can be inserted into mammalian expression vectors containing the genes encoding human immunoglobulin constant regions. We have recently developed a series of constructs that permit the rapid conversion of phage display library-derived scFv antibody fragments to fully human monoclonal antibodies of each immunoglobulin isotype and subclass (Huls et al. 1999; Boel et al. 2000). Transfected cell lines harboring these constructs produce human monoclonal antibodies in vitro that are correctly assembled and glycosylated.

It has been shown that treatment with monoclonal antibodies or antibody-derivatives directed against tumor surface antigens is a rational strategy in tumor immunotherapy. One route of therapy in cancer is aimed at recruiting the humoral and/or cellular arms of the immune system to eradicate tumor cells. To that end, a variety of approaches has been tested in in vivo and in vitro systems including unconjugated antibodies, bi-specific antibodies, immunotoxins, radio-labeled monoclonal antibodies, immunoliposomes and cytotoxic T lymphocytes (reviewed in Renner and Pfreundschuh 1995; Schneider-Godicke and Riethmuller 1995; Vile and Chong 1996; Maloney and Press 1998; Curnow 1997). For example, treatment of a large cohort of patients with resected Dukes C colorectal cancer with a murine monoclonal antibody against the Ep-CAM molecule expressed on colorectal tumor cells has been proven to be very effective. After 7 years of follow-up evaluation, overall mortality of patients was reduced by 32% and the recurrence rate was decreased by 23% (Riethmuller et al. 1998). In another example, a humanized monoclonal antibody against the HER2/neu receptor that is over-expressed on the tumor cells of 25-30% of patients with breast cancer, was shown to slow the progression of tumor growth and to increase the percentage of patients who experienced tumor cell shrinkage (Baselga et al. 1996). In another example, a chimeric monoclonal antibody against the CD20 antigen was shown to give a response in 48% of patients with relapsed low grade or follicular lymphoma (McLaughlin et al. 1998).

In the identified examples, treatment of patients with monoclonal antibodies is sufficient for clinical effect. Binding of the monoclonal antibody to the target antigen results in the recruitment of components of the complement system and effector cells of the immune system with Fc receptor for antibody constant regions that act in concert to kill the tumor cell. For reasons unknown, binding of a monoclonal antibody to a target antigen on the membrane of a cell does not always result in tumor cell killing. For some antigens, it is necessary to arm the monoclonal antibody with entities that kill the tumor cells via other mechanisms such as toxins or radioactive molecules.

In the identified approaches, the specificity of the monoclonal antibody employed is crucial. Ideally, the monoclonal antibody should specifically bind to tumor cells with minimal cross-reactivity with normal tissues. This criterium is not always met as illustrated by the chimeric anti-CD20 monoclonal antibody that was approved for clinical use (see, below). The CD20 antigen is expressed by B cell tumors but also by non-malignant immature and mature B lymphocytes. Despite this cross-reactivity with non-malignant B cells, patients receiving treatment with the chimeric anti-CD20 monoclonal antibody causes few side effects and the clinical success is considerable. It is clear that although several antibodies have been approved for clinical use (such as the anti-CD20 antibody) that there is a strong need for monoclonal antibody based treatments in which the antibody only and specifically targets the diseased cells.

### DISCLOSURE OF THE INVENTION

The present invention discloses methods and means to treat multiple myeloma (MM), which is a hitherto incurable neoplastic disease of the B-cell lineage, characterized by the presence of multifocal loci of monoclonal plasma cells in the bone marrow (BM). The disease can present itself with homogeneous serum immunoglubulin, osteolytic lesions, anemia, uremia, hypercalcemia, hyperviscosity, amyloidosis, secondary immunodeficiency and renal insufficiency. These clinical symptoms are dependend of the location, tumor load and the pathophysiology of malignant plasma cells. Conventional chemotherapy, like intermittent melphalan and prednisone, remains an unsatisfactory treatment of MM with a low response rate of about 50-60%, few complete remissions (CR<5%) and a median survival of only 30-36 months (Alexanian and Dimopoulos 1994; Boccadoro et al. 1997).

Allogeneic Bone Marrow Transplantation (Allo-BMT) may proof beneficial for myeloma patients when the observed graft versus myeloma effect can be maximally exploited and the problems of occurring graft versus host disease are circumvented. Encouraging results have been published of trials with high-dose chemo(radio) therapy and autologous stem cell transplantation (Tricot et al. 1995; Cunningham et al. 1994; Harousseau et al. 1995; Bjorkstrand et al. 1995; Attal et al. 1996). Specifically when applied early in the course of the disease in phase II studies dose intensification yielded 30-50% complete remissions. From a French collaborative study it was concluded that the most important prognostic factor in the autologous stem cell transplantation procedure is the achievement of a complete remission during initial chemotherapy (Harousseau et al. 1995). The median survival for patients responding to primary treatment was 54 months versus 30 month for the non-responders. Induction of CR is presumably the first step towards cure in MM. Nevertheless, patients in CR after autologous stem cell transplantation show a high relapse rate and there is no plateau in survival curves and therefore it seems unlikely that cure may be achieved by intensive therapy alone. Additional therapeutical strategies should thus be directed at eliminating residual tumor cells.

In one embodiment, the invention provides methods for selecting myeloma specific antigens and their specific interacting proteins. More in particular, the invention discloses binding molecules that selectively interact with tumor specific antigens, whereas the antigens are expressed on myeloma cells, other tumor cells and not on the normal CD46 positive cell types analyzed thus far. Antibody mediated therapies in myeloma have thus far been rather unsuccessful, partly because of absence of antigens with a plasma cell restricted expression pattern. However recently, vaccination experiments in a mouse model system using a tumor idiotype with comparable patho-biological features as human MM, suggest that the immune system can effectively be mobilized against myeloma tumor cells (King et al. 1998).

The invention further provides a novel MM tumor marker namely human CD46, which is also known as Membrane Cofactor Protein (MCP) and it provides more in particular, binding molecules that specifically interact with differentially glycosylated forms of human CD46. CD46 is a one- or two-band profile type 1 membrane protein of approximately 60.000 Dalton in molecular weight and is expressed on all nucleated cells. A soluble form of the protein was also found (Hara et al. 1992). The protein protects host cells from autologous complement attack and serves as a measles virus receptor, facilitating virus to cell and cell-to-cell attachment and fusion (Naniche et al. 1993; Dorig et al. 1993; Iwata et al. 1995). The fysiological role of CD46 is to protect the host cell from complement-mediated cell damage (Oglesby et al. 1992; Seya et al. 1990b). CD46 consists of four short consensus repeat domains, known as complement control protein (or CCP) repeats: a Ser/Thr-rich (ST) domain, a 13 amino acid unique sequence followed by a transmembrane (TM) portion and a cytoplasmic (CY) tail. Alternative splicing of CD46 transcripts causes different combinations of the exons of the gene encoding CD46, yielding a number of different isoforms (Liszewski et al. 1991). The amino terminus, identical for all isoforms, contains three potential N-glycosylation sites in CCP1, CCP2 and CCP4. The ST domain is extensively O-glycosylated. The glycosylation of CD46 was suggested to play a role in complement regulatory functions (Liszewski et al. 1998). To date six isoforms of CD46 have been identified in human cell lines, testis and placental cDNA libraries (Post et al. 1991).

Many tumor cells and cell lines express particularly high levels of complement regulatory proteins, like CD46, decay-accelerating factor (DAF, CD55) and protectin (CD59) (Seya et al. 1990a). It has been shown that human CD46 rather than CD55 is a key element in protection against complement activation (Van Dixhoorn et al. 2000). Overexpression of CD46 was observed in gastro-intestinal tumors, carcinomas of breast, cervix and endometrium, and hepatocellular carcinomas (Murray et al. 2000; Kinugasa et al. 1999; Schmitt et al. 1999; Thorsteinsson et al. 1998; Simpson et al. 1997). The difference in expression profiles of this membrane complement regulatory protein between normal and pathological tissues suggest resistance of tumor tissue to complement-mediated damage, thereby allowing tumor cells to escape from cytolysis and thus promoting tumor outgrowth.

One example of a successful tumor immuno-therapeutical approach is based on antibody and complement regulatory proteins. Overexpression of complement regulatory proteins that inhibit complement dependent cytotoxicity can result in a failure of monoclonal antibody therapy. This phenomenon is demonstrated in vitro by neutralization experiments using specific antibodies directed against complement regulator proteins. (Juhl et al. 1997; Jurianz et al. 1999). These studies that used breast carcinoma cells in combination with recombinant monoclonal antibody anti-HER2 or gastrointestinal cancer cells in combination with 17-1A anti-EpCAM revealed that the killing of tumor cells could be significantly increased by the use of antibodies directed against complement regulatory proteins, like CD46. So, anti-CD46 monoclonal antibodies can be very useful to overcome the limitation of the potential of monoclonal antibodies mediated by complement resistance.

In one aspect, the invention provides methods for determining differentially expressed, folded and/or post-translationally modified proteins on cells, in particular differentially glycosylated CD46 forms on tumor cells. The potential for structural diversity of glycans in metazoan cells is very large given the possible combinations of monosaccharides, linkages, branching, and variable lengths of glycan chains. The structural variability of glycans is dictated, among others, by tissue specific regulation of glycosyltransferase genes, acceptor and carbohydrate availability in the Golgi, compartimentalization, and by competition between enzymes for acceptor intermediates during glycan elongation (reviewed in Dennis et al. 1999). At any particular glycosylation site of a mature glycoprotein, a range of biosynthetically related glycan structures may be present. The prevalence of particular glycan structures on specific glycoprotein molecules can affect their functions, including half-life, localisation and biological activity. Aberrant glycosylation has been associated with tumor cells of epithelial origin. In fact, epitopes expressed by the MUC-I antigen expressed on epithelial tumors are targets for various forms of immunotherapy. Previous experiments have suggested that deveating glycosylation of membrane and secreted proteins may be a characteristic of plasma cells and the malignant cells in MM. An incompletely sialylated form of CD44 on a myeloma cell has been described and immunoglobulins produced by myeloma cells have a distinct oligosaccharide profile (Slupsky et al. 1993; Farooq et al. 1997). Proteoglycans of B lymphocytes undergo structural changes during B cell ontogeny that may correspond to the specific requirements of the respective microenvironment of the maturing cell (Engelmann et al. 1995). Nothing in these studies suggests that certain post-translationally modified variants of proteins can or have been used to obtain specific binding molecules according to the present invention.

An increasing number of diseases are being treated with chimeric-, humanized- or fully human antibodies that specifically recognize different kinds of disease associated molecular markers. The use of these markers as antigens for antibody-recognition is in many cases based on their respective over-expression on several cell lineages, such as the over expression of CD52 in T- and B-lymphocytes (targeted by the CAMPATH-1H antibody) and specific expression of the CD20 antigen in B-cells (targeted by the Rituximab antibody, also known as Rituxan). Both CAMPATH-1H and Rituximab antibodies are being used for the treatment of malignancies such as low-grade non-Hodgin's lymphoma. Remicade (Infliximab) is an approved antibody and is directed against over-expression of Tumor Necrosis Factor alpha (TNF-alpha) found in several tissues and is used for the treatment of Crohn's disease. Another example of an approved antibody that makes use of the over-expression of its specific antigen is Herceptin (Trastuzumab) that targets the over-expressed HER2/neu (or neu/erbB-2) antigen on breast tumor cells. HER2/neu is a proto-oncogen that due to amplification is found to be over-expressed in numerous malignant epithelial tumor cells. Clearly, each of these antigens or disease associated molecular markers is characterized in that the marker is the result of the tissue specific expression or over-expression of an mRNA encoding the molecular marker, either through differential RNA transcriptional levels resulting in differential protein levels between healthy an diseased cells or between cells from different tissues, or through differentially RNA splicing patterns resulting in different splice variants from one particular gene.

Different post-translationally modified forms of proteins can be present on different kinds of tissues and -cells. The CD55 (or Decay Accelerating Factor DAF) protein is a heavily glycosylated membrane protein for which different antibodies exist recognizing different subsets of the protein due to their glycosylation states. The inventors of the present invention realized that a wealth of disease associated molecular markers is waiting to be explored, which markers cannot be identified using conventional target-identification programs. The present invention fulfills in a need for methods and processes of obtaining this new type of molecular markers and developing medicines and therapies on the basis of these markers to diagnose, to prevent or to combat diseases with which the molecular markers are associated. Due to the present invention disease associated molecule markers can be identified and used for the development of medicines and therapeutic strategies against various diseases. The new type of disease associated molecular markers include, but are not limited to glycosylation variants, phosphorylation variants and conformational variants of otherwise known proteins and will for the purpose of this invention be referred to as “disease associated molecular markers”, or simply as “novel epitopes”.

The present invention discloses processes for identifying post-translationally modified disease associated molecular markers (also named novel epitopes), the post-translationally modified disease associated molecular markers being present on diseased cells in their post-translationally modified disease associated form. The processes make use of phage display libraries displaying binding molecules, the binding molecules preferably being antibodies or antibody fragments. The processes according to the invention make further use of cell sorting techniques and fluorescence based parameters.

The invention also provides the identified post-translationally modified disease associated molecular markers as well as the binding molecules that bind to it. In a preferred embodiment, the present invention provides novel binding molecules such as scFv fragments or fully human IgG molecules that bind the human CD46 protein specifically present in a specific glycosylation state on diseased cells. In another aspect of the invention the binding molecules are characterized, processed and recombinantly expressed in mammalian cells, preferably human cells. The invention also provides such cells, comprising a nucleic acid according to the invention encoding the binding molecule. Purified binding molecules according to the invention are used for the preparation of diagnostic tools, such as kits or medicaments for the treatment of diseases, such as neoplastic diseases as cancer (e.g., colorectal cancer, MM and breast tumors). Methods for the treatment of individuals suffering from or at risk of suffering from a disease, comprising administering and the use of the binding molecules of the invention are also provided.

### BRIEF DESCRIPTION OF THE FIGURES

FIG. 1 Various forms of recombinant monoclonal antibodies based on murine antibodies (Chimeric and Humanized) and fully human monoclonal antibodies derived from transgenic mice or phage display libraries (Human).

FIG. 2 Steps in selection of phages binding to subpopulations of cells using flow cytometry. I. A heterogeneous mixture of cells is incubated with the phage library. Non-binding phages are removed by washing. II. Cells with bound phages are stained with fluorochrome-labeled monoclonal antibodies and cells of interest are isolated using a cell sorter. III. Phages are eluted from the isolated cells and used to infect bacteria. Phage antibodies are isolated from single bacteria. IV. Phage antibodies are finally used in flow cytometric and immuno-histochemical analysis to assess the tissue and cellular distribution of the target antigen.

FIG. 3 Sort critera for malignant plasma cells from bone marrow mononuclear cells from patients with MM based on high levels of CD38 expression and forward scatter/side scatter profile (Terstappen et al. 1990).

FIG. 4 Staining of cell suspensions prepared from blood, spleen, tonsil and adult bone marrow (ABM) of healthy individuals and bone marrow of patients with MM (MMBM) with a phage directed to thyroglobuline (control) or with K53.

FIG. 5 Amino acid sequence of scFv antibodies K19, K29 and K53 and VH and VL gene utilization (* Nomenclature according to Matsuda and Honjo (1996)).

FIG. 6 Staining of cell suspension prepared from bone marrow of patients with MM with K53 and positive cells were isolated by cell sorting and stained with May Grunwald Giemsa

FIG. 7 Expression cloning of K19 binding molecule using a human placental cDNA library cloned in baculovirus.

FIG. 8 Binding to glycosylation variants of CD46 by human monoclonal antibodies K19, K53 and the conventional murine anti-CD46 antibody J4.48, here depicted as CD46. Binding was determined in CHO cells, which were stably transfected with normal full length CD46 (BC1), with NQ replacements of CCP1 (NQ1)/ CCP2 (NQ2)/ CCP4 (NQ4) or with a CD46 deletion mutant in the serine/threonine/proline rich domain (ΔSTP).

FIG. 9 FACS analysis showing the binding of anti-CD46 monoclonal antibody K53/IgG1 and the negative control antibody GBS III/IgG1 to bone marrow cells isolated from myeloma patients co-stained with CD38 and CD138.

FIG. 10 Mean tumor size in NOD/SCID mice xenografted with colon carcinoma cell line LS174T and treated with K53/IgG1 or control GBS III human monoclonal antibodies.

FIG. 11 (A)-(E) Analysis of bone marrow cells derived from MM patients stained with fully human monoclonal antibody K53/IgG1 (here L53) and negative control antibody GBS III. The P numbers indicate the different patients. Cells were used fresh or after storage in liquid N₂.

FIG. 12 (A)-(C) Analysis of bone marrow cells derived from non-MM patients stained with fully human monoclonal antibody K53/IgG1 (here depicted as L53) and negative control GBS III (here GBS3). Abbreviations are explained in Example 7. The gates were set for different markers (CD20, CD45 and CD19) as indicated.

FIG. 13 Analysis of normal bone marrow cells (upper panel) and tonsil cells (lower panel) using fully human monoclonal antibody K53/IgG1 (here depicted as L53) and negative control GBS III.

FIG. 14 Analysis of normal monocytes (upper panel), T-cells (middle panel) and B-cells (lower panel) from normal blood using fully human monoclonal antibody K53/IgG1 (here depicted as L53) and negative control GBS III, selected for staining with their respective markers CD14, CD3 and CD19.

FIG. 15 Staining of human colon tumor tissue using fully human monoclonal antibody K53/IgG1 (left) and GBS III as a negative control (right).

FIG. 16 Effect of tunicamycin on binding of fully human monoclonal antibody K53/IgG1 (here indicated as K53, black bars), negative control antibody GBS III (open bars) and conventional murine anti-CD46 antibody J4.48 (here indicated as CD46, gray bars) to (A) LS174T colon tumor cells and on (B) T47D breast cancer cells. (C) Effect of swainsonine on binding of the antibodies mentioned in (A) and (B) to LS174T colon tumor cells.

FIG. 17 in vitro killing assay using fully human monoclonal antibody K53/IgG1 on LS174T colon tumor cells as target cells in the presence of whole blood. Black bars represent cytotoxicity detected after incubation with K53/IgG1; open bars indicate cytotoxicity detected after incubation with negative control GBS III.

FIG. 18 Mean tumor size in Balb/c (nu/nu) mice xenografted with the human colon carcinoma cell line LS174T and treated with fully human monoclonal K53/IgG1 or control antibodies GBS III and UBS-54 antibodies on day 1, 3 and 6 (Group A). All mice were included. When no tumor developed, the tumor size was adjusted to 0.

FIG. 19 Mean tumor size in Balb/c (nu/nu) mice xenografted with the human colon carcinoma cell line LS174T and treated with fully human monoclonal antibody K53/IgG1 or control antibodies GBS III and UBS-54 on day 6, 9 and 12 (Group B). All mice were included. When no tumor developed, the tumor size was adjusted to 0.

FIG. 20 Mean tumor size in Balb/c (nu/nu) mice xenografted with the human colon carcinoma cell line LS174T and treated with fully human monoclonal antibody K53/IgG1 or control antibodies GBS III and UBS-54 on day 9, 12 and 15 (Group C). All mice were included. When no tumor developed, the tumor size was adjusted to 0.

FIG. 21 Mean tumor size in Balb/c (nu/nu) mice xenografted with the human colon carcinoma cell line LS174T and treated with fully human monoclonal antibody K53/IgG1 or control antibodies GBS III and UBS-54 on day 1, 3 and 6 (Group A). Only tumor bearing mice were included.

FIG. 22 Mean tumor size in Balb/c (nu/nu) mice xenografted with the human colon carcinoma cell line LS174T and treated with fully human monoclonal antibody K53/IgG1 or control antibodies GBS III and UBS-54 on day 6, 9 and 12 (Group B). Only tumor bearing mice were included.

FIG. 23 Mean tumor size in Balb/c (nu/nu) mice xenografted with the human colon carcinoma cell line LS174T and treated with fully human monoclonal antibody K53/IgG1 or control antibodies GBS III and UBS-54 on day 9, 12 and 15 (Group C). Only tumor bearing mice were included.

FIG. 24 Cloning procedures for the construction of pCD46-3000/Neo.

FIG. 25 Schematical representation of pCD46-3000/Neo.

FIG. 26 Ascending expression levels of 138 positive PER.C6 clones expressing recombinant K53/IgG1 after stable integration of pCD46-3000/Neo and subsequent G418 selection.

FIG. 27 SDS-PAGE and subsequent Coomassie staining of recombinant fully human monoclonal antibody K53/IgG1 produced on PER.C6 and purified over protein-A. The upper panel shows a reducing gel separating the heavy and light chains and the lower panel shows a non-reducing gel leaving the heavy and light chains linked. Five different clones are shown.

FIG. 28 Reducing gels showing a set of purified recombinant K53/IgG1 monoclonal antibodies produced on different PER.C6 clones. Separated proteins were stained with Coomassie Brilliant Blue.

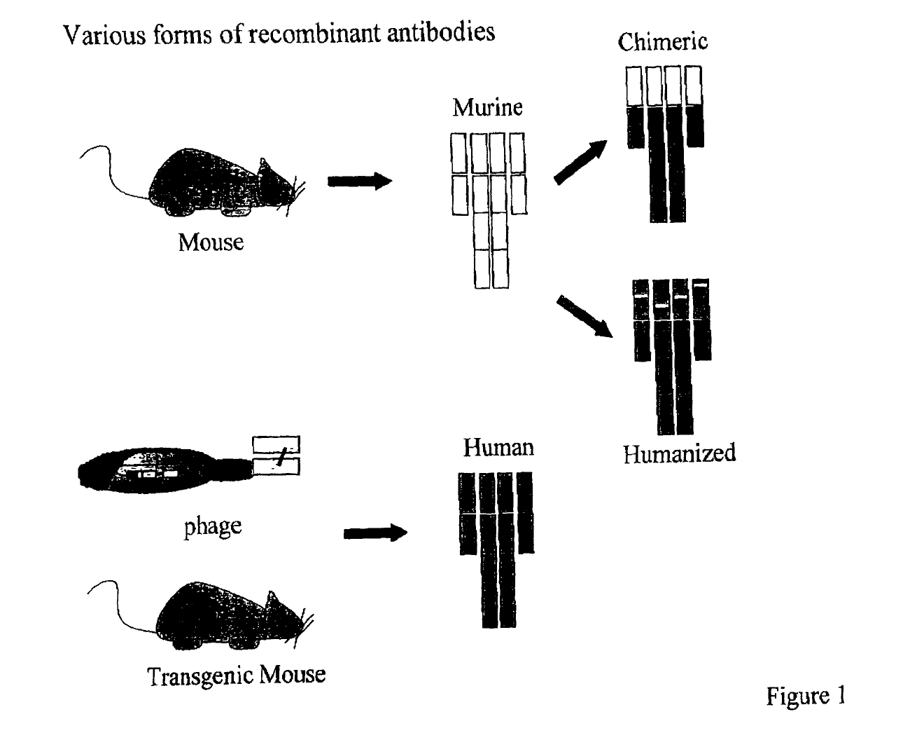

### DETAILED DESCRIPTION

The present invention provides a process for identifying a disease associated molecular marker associated with a subset of cells comprising the steps of:

a) incubating cells of a species with a library of binding molecules, combined with an incubation with diseased cells of the species;

b) obtaining from the incubation, a collection of diseased cells essentially free from non-diseased cells, by sorting the collection of diseased cells from non-diseased cells according to parameters which distinguish between the collection of diseased cells and the non-diseased cells;

c) obtaining binding molecules from the collection of diseased cells;

d) selecting from the obtained binding molecules, an individual binding molecule capable of preferential binding to the diseased cells as compared to binding to the non-diseased cells;

e) identifying a molecular marker which, in its disease associated form, binds to the individual binding molecule selected under step d), the molecular marker being associated with the collection of diseased cells obtainable according to step b); and

f) establishing that the disease associated form has acounterpart associated with non-diseased cells wherein the counterpart is less capable of binding the individual binding molecule. In a preferred embodiment the process further comprises the step of establishing that the counterpart and the disease associated form differ in at least one post-translational modification. Genetic differences (i.e. where the modification is the result of a change in RNA or DNA encoding the marker) can typically also be found by other means. However, the present invention has clear advantages for identifying post-translationally modified disease associated markers. In a preferred embodiment the post-translational modification comprises a glycosylation modification. This type of modification is relatively easy to identify and isolation binding molecules for. In one embodiment the process further comprises the steps of

recovering the individual binding molecule which binds to the molecular marker in its disease associated form; and

characterizing the individual binding molecule.

In another aspect, the invention provides for a process for identifying a binding molecule capable of binding a subset of diseased cells comprising the steps of:

a) incubating cells of a species with a library of binding molecules, combined with an incubation with diseased cells of the species;

b) obtaining from the incubation, a collection of diseased cells essentially free from non-diseased cells, by sorting the collection of diseased cells from non-diseased cells according to parameters which distinguish between the collection of diseased cells and the non-diseased cells;

c) obtaining binding molecules from the collection ofdiseased cells;

d) selecting from the obtained binding molecules, an individual binding molecule capable of preferential binding to the diseased cells as compared to binding to the non-diseased cells;

e) recovering the individual binding molecule selected in step d);

f) establishing that the individual binding molecule preferentially binds to a molecular marker in its disease associated form, the molecular marker in its disease associated form being associated with the diseased cells, the molecular marker further having a counterpart associated with non-diseased cells wherein the counterpart is less capable of binding the individual binding molecule. Preferably the process further comprises the step of establishing that the counterpart and the disease associated form differ in at least one post-translational modification. Preferably, the post-translational modification comprises a glycosylation modification.

A post-translationally modified molecular marker does not need to comprise post-translational modifications in the disease associated form. What is needed is a difference in post-translational modification between the form in a normal cells and a disease associated form. A molecular marker of the invention preferably does not comprise amino-acid differences between it's diseased associated form and it's counterpart in non-diseased cells.

Examples of post-translationally modified disease associated molecular markers or novel epitopes as used herein can be, but are not limited to, extra-cellular proteins or cell surface proteins, or parts thereof, that have undergone conformational or configurative changes due to differential N-glycosylation and/or O-glycosylation, phosphorylation, bridging (e.g. di-sulphide bridges), gamma-carboxylation and gamma-hydroxylation depending on the diseased state of the cells that they are associated with. “Disease associated”, as used herein, means that the marker is secreted by, bound by, attached to or targeted to a diseased cell; the cell being a diseased cell involved in disease. Of course, it is also possible to use equivalents of cells in a process of the invention. For instance, cells that are fixed or, fragments of cells or cell extracts or dispersions. Equivalents typically comprise complex mixtures derived from intact cells, these complex mixtures may be partly purified. However, purified protein fractions are typically not suitable equivalents for cells in the present invention.

In a preferred embodiment, the library of binding molecules comprises a phage antibody display library. However, the library of binding molecules can also comprise but are not limited to, small molecules, peptides, polypeptides or other proteinaceous molecules. In a more preferred embodiment the phage antibody display library comprises at least 1×10⁸ specificities. The phage antibody display library preferably display scFv or Fab fragments on the surface of bacteriophage particles. Preferably the process of the invention is a process, wherein the sorting is conducted using a fluorescent activated cell sorter (FACS) and wherein the parameters are fluorescence based parameters. In another aspect of the invention, the diseased cells, used in a process according to the invention, are present in a cell population derived from mammalian species suffering from diseases such as cancer (tumor cells, also referred to as neoplastic cells), diabetes, Alzheimer's disease, multiple sclerosis, rheumatoid arthritis, inflammatory disease or viral infections. In an even more preferred embodiment the diseased cells are MM cells, breast tumor cells or colon carcinoma cells. The mammalian species can be, but is not limited to, human. Following the sorting of cells, the binding molecule and/or the post-translationally modified disease associated molecular marker are recovered from the diseased cells that are identified in the sorting by conventional methods known in the art and as described in the examples disclosed herein. Preferably, the sorting comprises sorting using a fluorescent activated cell sorter.

In a preferred embodiment, the binding molecule comprises an scFv antibody fragment. An scFv can be used for specificity studies, while still being present on the phage particle. It can also be used purified form. Association with phage allows characterization of its encoding DNA sequence. The DNA can be used to construct or to form a full sized fully human immunoglobulin molecule that can be cloned into mammalian expression vectors and expressed in eukaryotic cells. The produced immunoglobulin can be purified from the medium and subsequently used in experimentation (as explained in the examples), and in the preparation of medicaments and/or diagnostic compounds. In a preferred aspect the eukaryotic cells are mammalian cells. Even more preferred are human cells for the expression of the fully human antibody comprising the binding moiety of the initially identified binding molecule.

In one aspect of the invention, the post-translationally modified disease associated molecular marker comprises a glycosylation variant of a cell surface protein, such as the CD46 protein. However, the post-translationally modified disease associated molecular marker can also comprise phosphorylation or conformational variants of a (cell surface) protein, or a protein that is released from the cell but nevertheless associated with a diseased cell according to the invention.

The present invention provides post-translationally modified disease associated molecular markers and binding molecules obtainable by a process according to the invention.

In a preferred embodiment, the post-translationally modified disease associated molecular marker comprises a post-translationally modified CD46 protein.

In another preferred embodiment, the binding molecule binds the post-translationally modified CD46 protein present on a subset of cells. The binding molecule binding the post-translationally modified CD46 protein present on a subset of cells does not bind, or does bind to a significant reduced level to a post-translationally modified CD46 variant present on non-diseased cells.

The present invention provides among others, novel disease associated molecular markers, methods for finding the novel epitopes and binding molecules capable of binding to the novel epitopes. Preferably, the markers and/or the binding molecules are obtained by a method of the invention. A disease associated molecular marker may comprise of one proteinaceous molecule. However, the marker may also be part of a complex. The epitope on the disease associated marker of the invention can be present or provided by one proteinaceous molecule. However, it can also be formed in combination with one or more other molecules. However, preferably, the epitope is formed by or present on one proteinaceous molecule.

In a preferred embodiment, the invention provides a binding molecule capable of specifically binding to an epitope present in a subset of CD46 proteins. The binding molecule is capable of distinguishing between CD46 proteins belonging to the subset and CD46 proteins not belonging to the subset. The binding molecule can thus be used to type samples containing CD46 protein, for example in diagnosing diseases. This property is also useful in, for instance, CD46 purification strategies.

CD46 is a protein that is widely expressed on many different cell types and tissues. In a preferred embodiment a binding molecule of the invention is capable of binding to a subset of CD46 expressing cells. Preferably, the binding is specific for the subset of CD46 expressing cells. A cell belonging to the subset of cells comprises a detectable amount of CD46 protein comprising the novel epitope. This cell can also comprise CD46 protein not comprising the novel epitope. A CD46 positive cell that does not belong to the subset contains CD46 protein not comprising the novel epitope. Typically, this cell does not comprise detectable levels of CD46 protein comprising the novel epitope, however, this may not always be true. With essentially specifically binding to a subset of CD46 positive cells is meant that the number of CD46 proteins comprising the novel epitope on a cell not belonging to the subset, is too low to be detected or to exert a biological effect upon binding of a binding molecule of the invention.

Preferably, a cell belonging to the subset of cells comprises a neoplastic cell. It has been found that many CD46 proteins of neoplastic cells comprise at least one novel epitope that is essentially not present on CD46 proteins expressed on normal CD46 positive cell types, thus far analysed. Preferably, the neoplastic cell is derived from a hemopoietic cell, a cervix cell, a colon cell, a kidney cell or a liver cell. In a particularly preferred embodiment the neoplastic cell is derived from a B-cell. More preferably the neoplastic cell comprises a MM cell.

A novel epitope according to the invention can consist of any (combination of) substance(s). Typically, a novel epitope is formed by an amino acid sequence, a sugar or lipid moiety, a (partly) post-translational modification or a combination of these elements. Post-translational modifications can be among other events the result of differential phosphorylation, differential glycosylation, conformational changes, such as di-sulphide bridging, multimerization in which two or more monomeric proteins form a novel epitope that can interact with a binding molecule of the invention, and the like. The modifications can make up the epitope in a form that can interact with a binding molecule. An example of a mycoprotein that is expressed on certain diseased cells and that has a different post-translational modification is MUC-1. When the epitope is not present, one or more of the substances making up the epitope are not available for binding to a binding molecule. The one more substances making up the epitope do not have to be absent from the molecule. All the substance(s) can still be present in or on the protein, however in this case, the one or more substances are present in a form that does not allow binding of a binding molecule of the invention. Typically, this is the case when the binding molecule is prevented from binding due to sterical hindrance and/or due to a conformational change in the protein leading to a different distribution of the one or more of the substance(s) in the protein.

In one aspect, the invention provides different glycosylation forms of CD46. At least one variant glycosylation form of CD46 comprises a novel epitope that is expressed on e.g. MM cells, whereas the novel epitope is not expressed by normal CD46-positive cell types thus far analyzed. A similar epitope is present in many other neoplastic cells (described below). The CD46 epitope present on MM cells thus is a suitable marker for at least some types of tumor cells. The invention therefore provides the use of a binding molecule of the invention for the typing of a CD46 positive cell. Preferably, the use comprises a diagnostic use. Even more preferably, the use comprises a preventive and/or curative therapeutic use.

To date, CD46 proteins of various animal species have been isolated. A person skilled in the art is well capable of identifying CD46 protein in species from which the CD46 protein is not yet determined. A suitable strategy is to use the information from an identified CD46 in an evolutionary closely related species. This can be the nucleic acid and/or amino acid sequence (for homology hybridization of nucleic acid libraries under more or less stringent conditions or nucleic acid amplification strategies using primers for conserved evolutionary conserved regions). Alternatively, antibodies specific for conserved parts of a CD46 molecule of an evolutionary related species can be used to identify a CD46 protein in another species. In a preferred embodiment the CD46 protein comprises a mammalian CD46 protein. More preferably, the CD46 protein comprises a human CD46 protein.

A binding molecule of the invention can be any type of binding molecule known in the art. A binding molecule is capable of specifically binding a novel epitope, meaning that the binding molecule does not bind efficiently to proteins not comprising the novel epitope. Many different types of binding molecules are known in the art. Examples of binding molecules according to the invention are, but are not limited to, small molecules, lectins, peptides, polypeptides and proteins such as antibodies or immunoglobulins (Ig) or Ig-like molecules. In a preferred embodiment, a binding molecule of the invention comprises an antibody or a functional part or derivative thereof. Suitable parts and/or derivatives of antibodies are Fab fragments, single chain Fv fragments, CDR domains, single chain Fab fragments or variable regions of the antibody molecule. An antibody may be produced or first generated by a B-cell. However, currently, many different ways of producing artificial antibodies are known in the art. An artificial antibody comprises a similar structure as a classical antibody. Such artificial antibodies can for instance be generated by in vitro assembly of amplified nucleic acid coding for various parts of an antibody. A functional part of an antibody comprises at least a part involved in epitope binding. A functional part of an antibody typically comprises the same epitope binding capacity in kind not necessarily in amount. A person skilled in the art is well capable of altering parts of the amino-acid sequence of an antibody without essentially affecting the binding capacity of the antibody. Alterations can comprise deletions, insertions, amino-acid substitutions or a combination of these alterations. Such derivatives of antibodies are also part of the invention.

In the art it has been observed that antibodies comprising a substantial amount of murine sequences do not perform very well in humans. Typically, the pharmacodynamics of such molecules are not comparable to the dynamics of a fully human or humanized antibody. Another disadvantage is that murine protein sequences are capable of eliciting a strong immune response in humans thereby further decreasing the utility of such murine sequence containing antibodies in humans. Preferably, an antibody of the invention is a human antibody or a humanized antibody. Such antibodies closely resemble normal human antibodies and have similar pharmacodynamics upon administration to humans.

In one embodiment, a binding molecule of the invention comprises a tag. A tag can be used for detection purposes. Alternatively, a tag can comprise a toxic substance. A toxic substance can enhance removal of targeted cells from the body. In one embodiment the toxic substance comprises a toxin and/or a radioactive substance.

In another embodiment, the invention provides a method for the treatment of an individual suffering from or at risk of suffering from a disease, comprising administering to the individual a therapeutically acceptable amount of a binding molecule of the invention. Administration can be used to facilitate removal of, undesired cells that cause at least part of the disease from the body of the individual. Such cells may be present in the body upon administration or the individual may be at risk of comprising the undesired cells. In a preferred embodiment the disease is a neoplastic disease. The invention also provides the use of a binding molecule of the invention for the preparation of a medicament for the treatment of certain diseases. Preferably, the medicament is used for the treatment of neoplastic disease. A binding molecule of the invention may be used in conjunction with other methods of treatments or medicaments. According to another embodiment, the other treatment or medicament comprises another binding molecule comprising a specificity for another epitope. Preferably, the another epitope is present on a CD46 expressing cell. Since CD46 is involved in the complement pathway, a molecule of the invention can be used to modulate complement mediated effects of the antibody specific for the another epitope. Modulation can comprise stimulation or inhibition of complement activity toward cells capable of binding both, a binding molecule of the invention and the antibody specific for the another epitope.

In one aspect of the invention, a binding molecule of the invention is used to type a cell. Now that different forms of CD46 can be detected it is possible to use this property in detection methods. In a non-limiting example a binding molecule of the invention is used to determine whether cells in a sample comprise neoplastic cells. Preferably, the neoplastic cell comprises a MM cell. In another aspect the invention provides the use of an epitope expressed on a subset of CD46 expressing cells as a marker for tumor cells. The invention therefore further provides a kit comprising at least a binding molecule of the invention. The kit preferably, further comprises a buffer suitable for allowing specific binding and/or means by which the binding can be detected, such as a fluorescence marker.

In yet another aspect, the invention provides a nucleic acid encoding a binding molecule according to the invention, or a part involved in CD46 binding of such a molecule. Such nucleic acid can be obtained in various ways. One non-limiting example is amplification of nucleic acid encoding the binding molecule from a cell expressing the molecule. In many methods for the generation of binding molecules and particularly for artificial antibodies, nucleic acid coding for the binding molecule is readily available.

A binding molecule of the invention can be produced in variety of ways. In a preferred embodiment a molecule of the invention is produced by a cell comprising a nucleic acid encoding the binding molecule. Preferably, the cell is a primate-, a rodent-, a bird- or a plant cell. Preferably, the cell is human cell. Human cells and the closely related primate cells have very similar post-translational modification machineries. In this way a binding molecule of the invention can be provided with human-like and more preferably, human post-translational modifications such as glycosylation. Such human type modification is less immunogenic in humans than modifications introduced by cells of other species, thus leading to a better efficacy of the treatment. In a preferred embodiment the cell further comprises a means for the conditional expression of a nucleic acid of interest. In this way, expression of a proteinaceous binding molecule of the invention can be induced at times when expression is desired. This is advantageous when expression of the proteinaceous binding molecule interferes with a function of the cell. By essentially avoiding expression during normal culture of the cells, the cells can be cultured and expanded normally before the condition for expression of the nucleic acid is fulfilled. A preferred system for the conditional expression of nucleic acid of interest comprises a tetracycline responsive expression system. The invention therefore also provides a cell comprising a nucleic acid encoding a binding molecule of the invention, the cell preferably further comprising a tetracycline responsive molecule capable of influencing expression of a promoter.

In a preferred embodiment, the cell comprises nucleic acid encoding an early protein of adenovirus or a functional part, derivative and/or analogue thereof. Such a cell is capable of producing more functional binding molecule per time unit. In a preferred embodiment the early protein comprises adenovirus E1 or a functional part, derivative and/or analogue thereof. Adenovirus E1 comprises transcriptional activation properties and generally has the effect of enhancing protein production in a cell. In a preferred embodiment the adenovirus early protein comprises adenovirus E2A or a functional part, derivative and/or analogue thereof. E2A in a cell has a protein production enhancing effect. Derivatives of E1 or E2A can be generated in various ways. One non-limiting way is through amino-acid substitution. A functional part and/or derivative of adenovirus E1 or E2A comprises the same protein production enhancing qualities in kind not necessarily in amount. Many viruses use proteins with similar protein production enhancing qualities as adenovirus E1 or E2A. Such molecules form suitable analogues of E1 or E2A.

In another aspect, the invention provides a plant or non-human animal comprising a cell capable of producing a binding molecule of the invention. Typically, though not necessarily such a plant or non-human animal is transgenic for a nucleic acid encoding a binding molecule of the invention. In one embodiment the animal comprises a human immunoglobulin locus or a functional part thereof.

In yet another aspect, the invention provides a gene delivery vehicle comprising a nucleic acid encoding a binding molecule according to the invention. Such a gene delivery vehicle can be used to target delivery of the nucleic acid contained in the gene delivery vehicle to diseased cells expressing a post-translationally modified protein belonging to a subset of proteins, such as the CD46 protein.

In yet another aspect, the invention provides a method for selecting a binding molecule capable of binding specifically to a epitope on a protein wherein the epitope is present on a subset of cells expressing the protein, the method comprising providing a collection of cells comprising cells of one type, with a library of proteinaceous binding molecules and selecting from binding molecules bound to the collection of cells at least one binding molecule capable of binding to the novel epitope.

The present invention demonstrates that molecular markers are present in disease associated, and preferably post-translationally modified, forms on the surface of diseased cells. These molecular markers can be identified and specifically targeted by binding molecules of the invention. More in particular, the invention demonstrates that tumor cells, for example but not limited to MM cells, express certain types of glycosylated forms of CD46 proteins, which are not expressed on non-tumor cell types. The invention makes use of this feature and provides also a number of binding molecules and more in particular fully human monoclonal antibodies that specifically interact with the differentially glycosylated human CD46 proteins. In a particularly preferred embodiment of the invention, these human monoclonal antibodies are used to diagnose, prevent and/or treat different kinds of human malignancies, in particular MM.

TABLE I
Binding capacity of K19, K29, K53 and tyroglobuline
(control) to different tumor cell lines analyzed by FACS.

| cell line | control | K19 | K29 | K53 |
|---|---|---|---|---|
| HeLa | 4 | 33 | 62 | 55 |
| HepG2 | 5 | 55 | 105 | 93 |
| MCF-7 | 6 | 89 | 128 | 157 |
| LS174T | 6 | 48 | 56 | 125 |
| COS-7 | 8 | | | 7 |

TABLE II
Complement-dependent cytotoxicity (CDC), antibody-dependent
cellular cytotoxicity (ADCC) and complement plus antibody-
dependent cellular cytotoxicity (CDCC) using human K53/IgG1,
K29/IgG1 and GBS III human monoclonal antibodies (20 μg/ml).
The numbers are expressed as percentage of cytotoxic LS174T
colon cancer cells and are the mean of 8 different donors.

| huMabs | control | CDC | ADCC | CDCC |
|---|---|---|---|---|
| — | 9.9 | 7.7 | 10.1 | 8.9 |
| GBS III | 8.5 | 6.2 | 11.6 | 13.2 |
| K53/IgG1 | 10.1 | 4.9 | 37.2 | 33.7 |
| K29/IgG1 | 9.9 | 4.5 | 20.7 | 18.9 |

TABLE III
Staining of bone marrow cells from multiple myeloma patients
using fully human monoclonal antibody K53/IgG1.

| Code | Sex | Date of Birth | Source | Disease Status | % Plasma cells¹ |
|---|---|---|---|---|---|
| P1 | f | 16/03/49 | fresh | primary | 30 |
| P2 | m | 14/02/47 | N₂ | primary | 50 |
| P3 | f | 12/01/49 | N₂ | refractory | 35 |
| P4 | m | 09/10/51 | N₂ | primary | 40 |
| P5 | m | 21/11/23 | N₂ | responsive | 60 |
| P6 | m | 31/03/55 | N₂ | refractory | 40 |
| P7 | f | 25/06/50 | N₂ | responsive | 20 |
| P8 | m | 07/12/41 | fresh | refractory | |
| P9 | m | 30/08/37 | N₂ | refractory | 65 |
| P10 | f | 13/05/24 | fresh | refractory | 7 |
| P11 | f | 18/06/43 | fresh | responsive | 2 |
| P12 | m | 12/07/47 | N₂ | refractory | 65 |
| P13 | f | 25/07/34 | fresh | refractory | |
| P14 | m | 07/12/41 | N₂ | refractory | 80 |

^{1 cytomorphological analysis}
Primary: at diagnosis; untreated
Refractory: relapse after chemotherapy (and in some instances BM transplant)
Responsive:
partial: increased % plasma cells in BM; presence of M component in serum; aberrant κ/λ ratio
complete: normal % plasma cells in BM; normal κ/λ ratio; no M component in serum

TABLE IV
Staining of leukemic tumors from non-MM patients using fully
human monoclonal antibody K53/IgG1. Abbreviations are
explained in example 7.

| Patient Code | Tumor type¹ |
|---|---|
| A1 | T-ALL |
| A2 | B-ALL |
| A3 | M0 myeloid |
| A4 | M0/M1 myeloid |
| A5 | M4 myeloid |
| A6 | M6 myeloid |
| A7 | CLL |
| A8 | NHL |
| A9 | NHL |
| A10 | NHL |
| A11 | NHL |
| A12 | NHL |
| A13 | NHL |

¹No staining of these tumors was observed

TABLE V
Binding of K53/IgG1 to myeloma-, leukemic- and solid tumor
cell lines determined by FACS as mean fluorescence
intensities. GBS III served as a negative control, while
J4.48 served as a positive, conventional anti-CD46 positive
control (here depicted as Anti-CD46, right columns).

| | | | |
|---|---|---|---|
| CELL LINE | GBS III | K53/IgG1 | Anti-CD46 |

MEAN FLUORESCENCE INTENSITIES OF ANTIBODIES ON
MYELOMA CELL LINES

| | | | |
|---|---|---|---|
| U266 | 8.7 | 52.9 | 366.8 |
| XG-1 | 16.2 | 183.2 | 554.6 |
| RPMI | 7.1 | 286.7 | 816.8 |
| DOX-6 | 5.8 | 71.3 | 450.3 |
| DOX-40 | 8.9 | 36.7 | 345.2 |
| XG+ (IL6 dep) | 29.5 | 429.8 | 956.9 |
| OPM-1 | 8.0 | 47.3 | 319.8 |
| L363 | 4.6 | 55.5 | 334.4 |
| UM3 | 5.7 | 36.8 | 471.8 |
| NCI | 4.0 | 9.2 | 187.8 |
| UM1 | 3.6 | 46.9 | 359.7 |
| TH | 5.1 | 23.5 | 183.2 |

MEAN FLUORESCENCE INTENSITIES OF ANTIBODIES ON
LEUKEMIC CELL LINES

| | | | |
|---|---|---|---|
| JURKAT | 3.9 | 11.9 | 219.4 |
| BJAB | 5.2 | 86.9 | 327.4 |
| RAJI | 3.0 | 37.8 | 246.2 |
| RAMOS | 5.4 | 6.2 | 179.1 |
| SP2.0 | 6.6 | 6.9 | 11.7 |

MEAN FLUORESCENCE INTENSITIES OF ANTIBODIES ON
SOLID TUMOR CELL LINES

| | | | |
|---|---|---|---|
| GLC1 | 4.6 | 8.8 | 80.8 |
| HEK293 | 4.5 | 5.5 | 70.3 |
| DLD-1 | 3.8 | 600.4 | 714 |
| HCT116 | 4.8 | 12.3 | 248.4 |
| HT29 | 3.1 | 96.5 | 224.1 |
| SW480 | 4.4 | 275.1 | 590 |
| LS174T | 5.3 | 622 | 380 |
| T47D | 4.0 | 1130 | 679 |
| MCF-7 | 6.8 | 824 | 650 |
| MDA-MB231 | 8.7 | 101 | 448 |
| COS | 5.7 | 6.5 | 3.4 |

TABLE VI
Staining of human adult normal tissue with fully human
monoclonal antibody K53/IgG1 in comparison to negative
control GBS III. The sex and the age of the respective
donors are also depicted.

| Tissue type | Sex | age | K53/IgG1 | GBS III |
|---|---|---|---|---|
| Adrenal | m | 25 | − | − |
| Heart | m | 26 | − | − |
| Brain | m | 26 | − | − |
| Kidney | m | 56 | − | − |
| Lung | m | 26 | − | − |
| Muscle | m | 26 | − | − |
| Liver | m | 30 | − | − |
| Spleen | f | 68 | − | − |
| Pancreas | m | 28 | − | − |
| Thalamus | m | 23 | − | − |
| Pituitary | m | 27 | − | − |
| Thyroid | m | 26 | − | − |
| Thymus | m | 23 | − | − |
| Colon | m | 28 | − | − |
| Breast | f | 45 | − | − |
| Stomach | m | 26 | − | − |
| Bladder | m | 28 | − | − |
| Esophagus | m | 70 | − | − |
| Tonsil | m | 50 | − | − |
| Thymus | f | 42 | − | − |
| Appendix | m | 60 | − | − |
| Lymph Node | m | 26 | − | − |
| Gallbladder | m | 25 | − | − |
| Prostate | m | 28 | − | − |
| Testis | m | 26 | − | − |
| Ovary | f | 50 | − | − |
| Small Intestine | m | 64 | − | − |
| Uterus | f | 43 | − | − |
| Placenta | f | 30 | − | − |
| Adipose | m | 64 | − | − |

TABLE VII
Staining of solid tumors using fully human monoclonal
antibody K53/IgG1 and as a negative control GBS III.

| Patient code | Tumor type | K53/IgG1 | GBS III |
|---|---|---|---|
| 99-14276 | Colon | + | − |
| 00-24912 | Colon | + | − |
| 00-70337 | Colon | − | − |
| 00-70345 | Colon | +/− | − |
| 99-60600 | Breast | + | − |
| 99-190100 | Breast | +/− | − |
| 00-17844 | Breast | +/− | − |
| 99-18193 | Kidney | − | − |
| 98-180700 | Kidney | − | − |
| 98-280700 | Kidney | − | − |

TABLE VIII
Scheme of antibody treatment in in vivo experiment using
Balb/c mice xenografted with LS174T cells.

| Group number | Treatment | Number of animals |
|---|---|---|
| Group A: | K53/IgG1 | 5 |
| day 1 (300 μg) | GBS III | 5 |
| day 3, 6 (150 μg) | UBS-54 | 5 |
| Group B: | K53/IgG1 | 5 |
| day 6 (300 μg) | GBS III | 5 |
| day 9, 12 (150 μg) | UBS-54 | 5 |
| Group C: | K53/IgG1 | 5 |
| day 9 (300 μg) | GBS III | 5 |
| day 12, 15 (150 μg) | UBS-54 | 5 |

TABLE IX
Mean tumor size in Balb/c (nu/nu) mice xenografted with the
human colon carcinoma cell line LS174T and treated with the
K53/IgG1 or control antibodies GBS III and UBS-54. All mice
(n = 5 mice × 2 flanks = 10) were included. When no tumor
developed, the tumor size was adjusted to 0. On day 13 one
GBS III treated mouse (bearing two tumors) from group B, and
one K53/IgG1 treated mouse (bearing one tumor) from group A
were killed.

| | | 9 | | | 13 | | | 15 | | | 17/18 | | |
|---|---|---|---|---|---|---|---|---|---|---|---|---|---|
| Day | huMab | mean | stdev | n | mean | stdev | n | mean | stdev | n | mean | stdev | n |
| Group A | GBS III | 14.3 | 22.2 | 10 | 87.4 | 153 | 10 | 148.8 | 235 | 10 | 377.8 | 491.1 | 10 |
| day 1,3,6 | UBS-54 | 2.8 | 6.7 | 10 | 29.9 | 65.8 | 10 | 26.4 | 55.9 | 10 | 110 | 233.1 | 10 |
| | K53/IgG1 | 2.8 | 6.7 | 10 | 19.2 | 52.8 | 10 | 1.9 | 5.3 | 8 | 63.8 | 168.5 | 8 |
| Group B | GBS III | 17.6 | 28.9 | 10 | 96.2 | 89.7 | 10 | 273.4 | 377.9 | 8 | 281.5 | 325.8 | 8 |
| day 6,9,12 | UBS-54 | 6.8 | 10 | 10 | 23 | 28.8 | 10 | 51.1 | 72.7 | 10 | 127.9 | 148.2 | 10 |
| | K53/IgG1 | 4.1 | 5.8 | 10 | 13.4 | 19.4 | 10 | 37 | 50.4 | 10 | 89.6 | 92.9 | 10 |
| Group C | GBS III | 4.2 | 7 | 10 | 61.2 | 155.5 | 10 | 95.1 | 132 | 10 | 156.9 | 261.9 | 10 |
| day 9,12,15 | UBS-54 | 4.6 | 11.2 | 10 | 28.5 | 44.9 | 10 | 45.3 | 66.2 | 10 | 74.2 | 120 | 10 |
| | K53/IgG1 | 4.4 | 6 | 10 | 32.1 | 51 | 10 | 50.6 | 64.4 | 10 | 60.5 | 118.5 | 10 |

TABLE X
Mean tumor size in Balb/c (nu/nu) mice xenografted with the
human colon carcinoma cell line LS174T and treated with the
K53/IgG1 or control antibodies GBS III and UBS-54. Only
tumor bearing mice were included. On day 13 one GBS III
treated mouse (bearing two tumors) from group B, and one
K53/IgG1 treated mouse (bearing one tumor) from group A were
killed.

| | | 9 | | | 13 | | | 15 | | | 17/18 | | |
|---|---|---|---|---|---|---|---|---|---|---|---|---|---|
| Day | huMab | mean | stdev | n | mean | stdev | n | mean | stdev | n | mean | stdev | n |
| Group A | GBS III | 17.8 | 23.7 | 8 | 109.3 | 165.3 | 8 | 165.3 | 243 | 9 | 377.8 | 491.1 | 10 |
| | UBS-54 | 14 | 8.5 | 2 | 148.5 | 61.5 | 2 | 132 | 17 | 2 | 550 | 70.7 | 2 |
| | K53/IgG1 | 14 | 8.5 | 2 | 64.1 | 90.8 | 3 | 7.6 | 10.4 | 2 | 255 | 318.2 | 2 |
| Group B | GBS III | 35.2 | 32.8 | 5 | 144.3 | 67.4 | 6 | 546.8 | 366.1 | 4 | 563 | 190.8 | 4 |
| | UBS-54 | 13.6 | 10.5 | 5 | 32.9 | 29.4 | 7 | 63.8 | 76.5 | 8 | 156.3 | 149.8 | 9 |
| | K53/IgG1 | 6.8 | 6.1 | 6 | 16.8 | 20.4 | 8 | 46.3 | 52.6 | 8 | 112 | 90.7 | 8 |
| Group C | GBS III | 10.5 | 7.5 | 4 | 129.4 | 207.9 | 5 | 174.2 | 135.2 | 5 | 224.1 | 292.1 | 7 |
| | UBS-54 | 6.6 | 13.1 | 7 | 47.5 | 50.4 | 6 | 75.3 | 71.6 | 6 | 123.7 | 136.3 | 6 |
| | K53/IgG1 | 7.1 | 6.3 | 5 | 53.5 | 57.6 | 6 | 84.3 | 63.7 | 6 | 96.8 | 142.1 | 5 |

TABLE XI
Recombinant fully human antibody K53/IgG1 production rates
in HEK 293 cells. Five separate harvests are depicted from
cell line L53-7 that gave the highest yields. The antibodies
were purified over protein-A and concentrations were
measured by ELISA.

| | | Concentration | Quantity |
|---|---|---|---|
| Harvest number | Fraction number | (mg/ml) | (ml) |
| H1 | 3, 4 | 0.45 | 7.0 |
| H2 | 2, 3, 4 | 0.5 | 10 |
| H3 | 3, 4 | 0.52 | 7 |
| H4 | 3, 4 | 0.63 | 7 |
| H5 | 3, 4 | 0.54 | 7 |

The invention is further explained by the use of the following illustrative examples.

### EXAMPLES

### Example 1

Isolation of Malignant Plasma Cell-specific Phage Antibodies K 19, K29 and K53 for a Semi-synthetic Phage Antibody Display Library

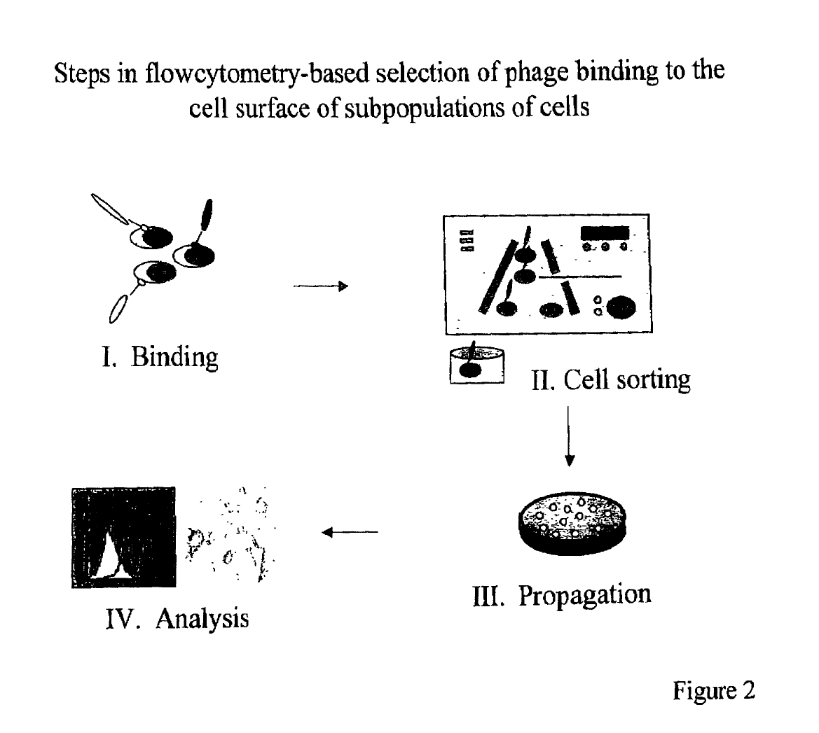

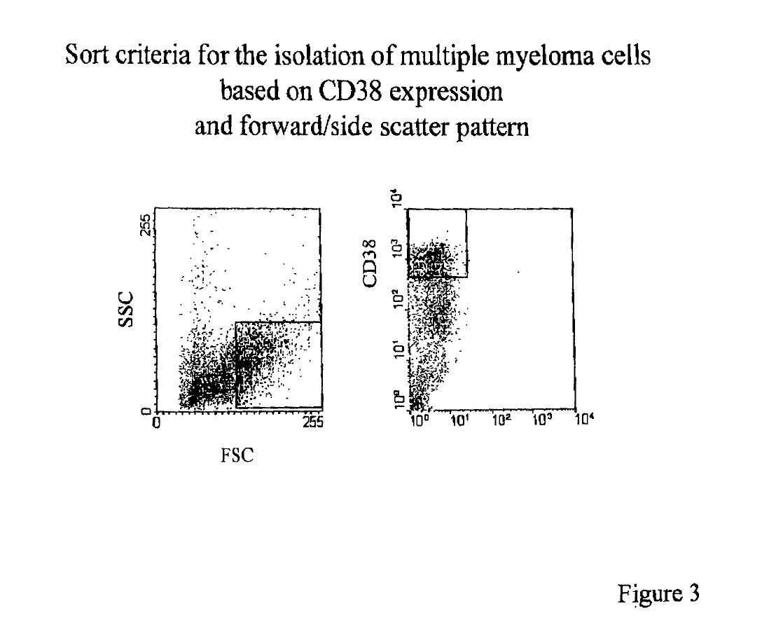

Phage antibody display in combination with flow cytometry (FIG. 2) was used to isolate human scFv antibody fragments that bind to malignant plasma cells. Details of this method have been described elsewhere (De Kruif et al. 1995a and 1995b). The preparation of cell suspensions from blood, tonsil, spleen and bone marrow from healthy individuals and multiple myeloma patients is described in Van der Vuurst de Vries and Logtenberg (2000). Mononuclear cells from these suspensions were isolated by Ficoll-Paque (Pharmacia) density centrifugation and subsequent washes in PBS plus 1% Bovine Serum Albumine (BSA) and used for FACS analysis as described (Van der Vuurst de Vries and Logtenberg 2000). The heterogeneous mixture of mononuclear cells of a patient with Multiple Myeloma was mixed with a phage display library of human scFv fragments, made essentially as described by De Kruif et al. (1995a and 1995b). Approximately 10¹³ phage particles were blocked for 15 min in PBS/1% BSA containing 4% low-fat milk powder. Bone marrow cells from a healthy donor were added to the blocked phages and the mixture was slowly rotated for 16 h at 4° C. Non-binding phages were washed away with ice-cold PBS/1% BSA and cells were subsequently stained with PE-labeled anti-CD38 antibodies. Malignant plasma cells were isolated by sorting on a FACSstarPlus (Becton Dickinson) based on high levels of CD38 expression and forward/side scatter profile (FIG. 3). It is known in the art that CD38 is highly expressed on these tumor cells and that they can be readily recognized by FACS analysis using staining procedures to detect CD38 proteins on their surface. FIG. 3 shows what population is preferably selected for the expression levels of CD38 and can be used thereafter in the methods of the present invention. Phages bound to the sorted malignant plasma cells were eluted by incubation at room temperature with 1.5 volume of 76 mM citric acid pH 2.5, followed by neutralization with 1.5 volume of 1 M Tris.HCl pH 7.4. The eluted phages were rescued by infection of XL1-blue F′ bacteria and propagated for a second round of selection. After three rounds of selection on the malignant plasma cells of three different patients with Multiple Myeloma, phages were prepared from individual bacterial colonies and used in immuno-fluorescent analysis as described (De Kruif et al. 1995a). Phage antibodies that bound to malignant plasma cells in the bone marrow of patients with Multiple Myeloma and that showed little staining (as performed by De Kruif et al. 1995a) of hematopoietic cells in other lymphoid organs were selected for further study. Three specifically and strongly interacting phage antibodies were identified and named K 19, K29 and K53.

### Example 2

Specific Interaction of K19, K29 and K53 Antibodies With Multiple Myeloma Cells

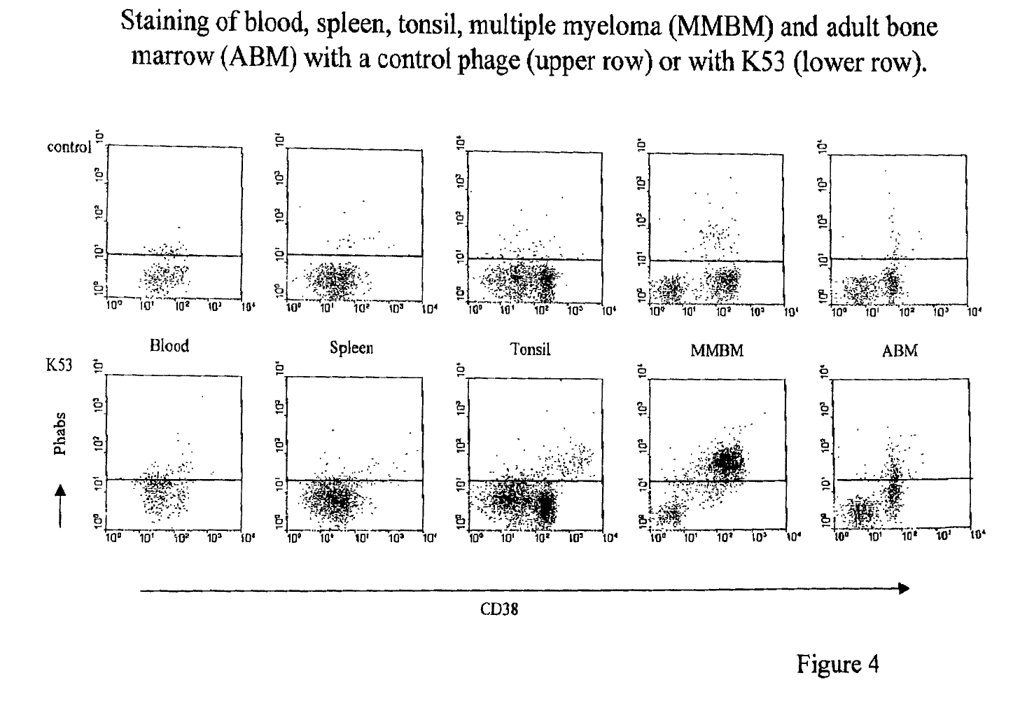

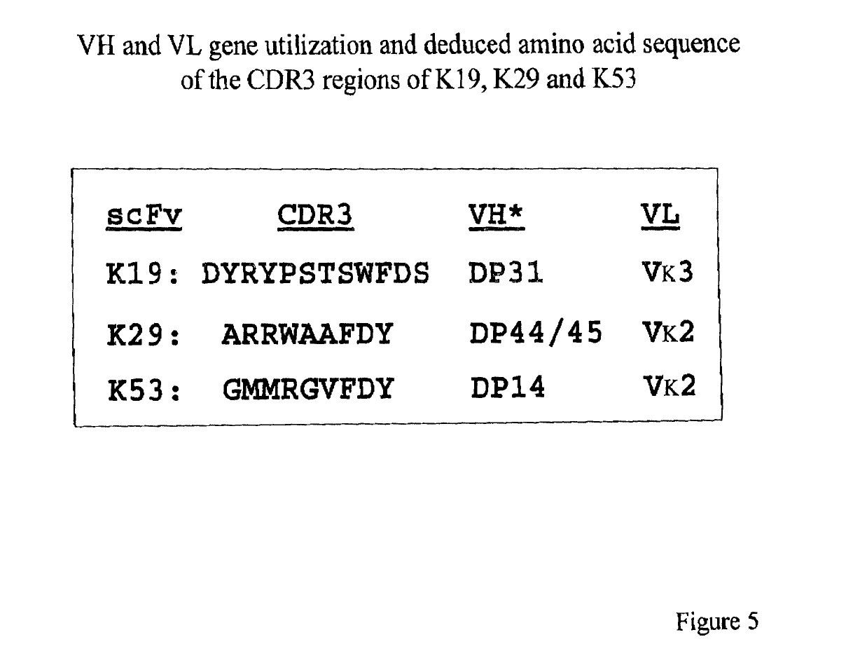

Cell suspensions were prepared from blood, tonsil, bone marrow and spleen of healthy individuals and subsequently stained with the three identified phage antibodies K19, K29 and K53. For this, cells were stained with myc-tagged single chain Fv fragments followed by anti-myc antibody (9E10) and fluorochrome labeled goat anti-mouse antibodies (Southern Biotechnology Associates) staining. The details concerning the use of these antibodies were described in De Kruif et al. (1995a and 1995b). Subsequently, binding capacity was analyzed by FACS. The scFv's only bound to the malignant plasma cells in the bone marrow of patients with multiple myeloma and to a small population of CD38 bright cells in tonsil and normal bone marrow. No staining of hematopoietic cells in spleen or blood was observed. FIG. 4 shows the binding of K53 to these cells as compared to a control phage antibody that is directed against thyroglobuline. The nucleotide sequence of the three scFv antibodies was determined and unveiled that K19, K29 and K53 were encoded by different immunoglobulin heavy and light chain variable genes (FIG. 5) and thus represented three independent scFv antibody fragments. The amino acid FDY motif that seems to overlap between these antibodies is present in many (also non-CD46 binding) antibodies. The determination of crucial residues involved in the CD46 specific interaction is investigated and described in more detail in Example 13.

### Example 3

Staining of Bone Marrow Cells From a Multiple Myeloma (MM) Patient by K53

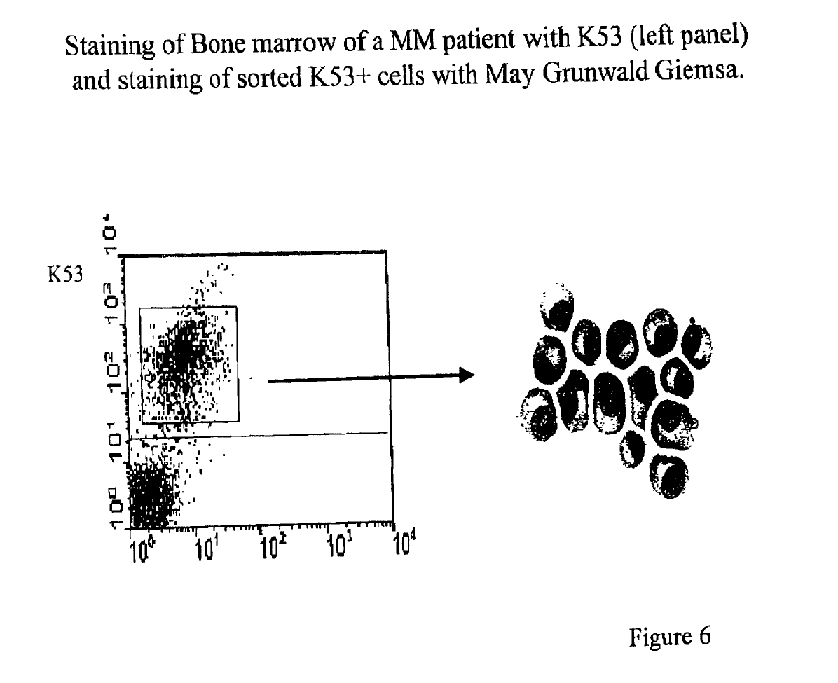

CD38 bright cells are cells that exhibit a very high CD38 expression (black dots in FIG. 3). Cells of this kind are found in bone marrow and tonsil cells and have been previously shown to represent plasma cells and precursors of plasma cells, or so-called plasma blasts (Terstappen et al. 1990). In order to confirm that the scFv antibody fragments recognized plasma cells, bone marrow cells from a MM patient were stained with scFv K53 and positive cells were isolated by cell sorting. For this, isolation, staining and cell sorting procedures were performed as described by De Kruif et al. 1995a) and Van der Vuurst de Vries and Logtenberg (2000). To spin preparations (80,000 cells were spun in 100 μl PBS at 500 rpm during 5 min) from the sorted cells were stained with May Grünwald Giemsa (Merck, 12% solution during 15 min in aquadest), washed with water, covered with a coverslip and cells were visualized by light microscopy. As shown in FIG. 6, sorted K53+bone marrow cells displayed a distinct plasma cell morphology.

### Example 4

Determination of the Human CD46 Protein as the Antigen That is Specifically Recognized by the MM Specific scFv Antibody Fragments

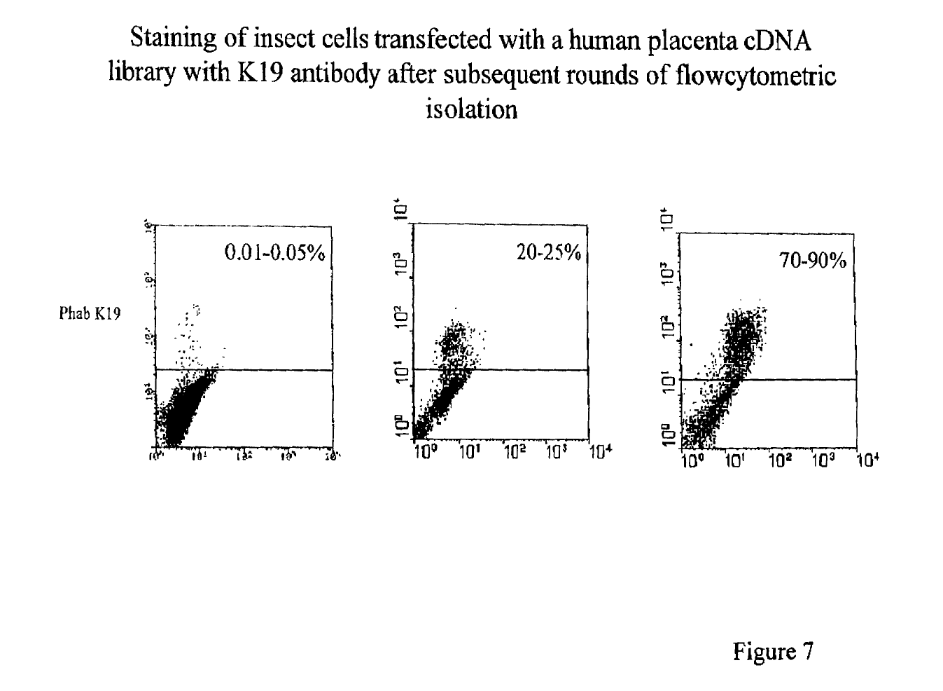

The phage antibody K19 was used to screen expression libraries using baculovirus in Sf9 cells using two human placental cDNA libraries cloned in baculovirus. The construction of the human placenta cDNA libraries in the baculovirus transfer vector pBacPAK9 (Clontech) is described in detail in Granziero et al. 1997). Two libraries were used, one containing cDNA inserts ranging in size from 1-3 Kb (1-3 Kb library) and the other containing cDNA inserts larger than 3 Kb (>3 Kb library). The titer of the libraries was determined using the BackPAK rapid titer kit (Clontech). The titer of 1-3 kb library was 1.1×10⁸ pfu/ml and the titer of the >3 kb library was 4×10⁷ pfu/ml. Sf9 insect cells (ATCC) were maintained at 27° C. in TC 100 medium supplemented with 5% FCS, pen/strep and 0.1% pluronic-F68 (Gibco). For screening of the cDNA libraries, 2×10⁶ Sf9 insect cells were washed and exposed to 1 ml of the 1-3Kb library (1.1×10⁸ pfu) or 1.5 ml of the >3 Kb library (6×10⁷ pfu). Cells were left for 1 h at room temperature before 5 ml medium was added. The cells were left for 48 h at 27° C. prior to staining with phage antibodies. By staining with phage antibodies, single positive cells were sorted. Subsequently the virus, which encodes the surface epitope was isolated by limiting dilution. Screening of the baculovirus expression library was performed 48 h after infection of Sf9 cells by incubating the cells with the scFv's using M13-biotynilated antibodies followed by a Phycoerythrin (PE)-labeled goat anti-mouse Streptavidine antibody. Single positive cells were sorted by FACSstar, propagated and used for a next round of selection. For this, single positive cells were mixed with fresh insect cells to propagate the baculoviruses. Supernatants of insect cells were used to infect fresh Sf9 insect cells and the entire procedure was repeated twice. The viruses, present in the supernatant of positive wells, were cloned by limiting dilution and the inserts were recovered by PCR and sequenced. Details of the cDNA library, the use of the library and virus identification by limiting dilutions are described by Granziero et al. (1997). The results of these experiments are shown in FIG. 7.

Nucleotide sequence analysis was subsequently performed by methods known to persons skilled in the art. The analysis of the cDNA inserts from clones obtained from two size-selected placental cDNA libraries, containing inserts smaller than 2 kb and inserts larger than 2 kb respectively, unveiled open reading frames that in both instances completely matched the reported nucleotide sequence of the human CD46 gene. Re-introduction of the human CD46 cDNA clones in Sf9 insect cells was performed using Lipofectamine transfection procedures (Life Technologies) and staining with scFv antibody fragments K19, K29 and K53 confirmed that all three scFv antibodies recognized the CD46 gene. Apparently, the transformed St9 insect cells are capable of expressing the specific human post-translationally modified CD46 form that is recognized by these scFv's and that is further investigated and described in Example 5.

### Example 5

Recognition of CD46 Subforms by the Multiple Myeloma Specific scFv Antibodies

CD46 is expressed on all nucleated cells including tumor cells; some tumor cells and cell lines express particularly high levels of CD46 (Kinugasa et al. 1999; Schmitt et al. 1999; Thorsteinsson et al. 1998; Simpson et al. 1997). The CD46 protein is also expressed on various tissues and organs and soluble forms of CD46 are present in most bodily fluids (Hara et al. 1992). The physiological role of CD46 seems to be to protect the host cell from complement-mediated cell damage (Oglesby et al. 1992; Seya et al. 1990a and 1990b) To date, six isoforms of CD46 have been identified in human cell lines, testis and placental cDNA libraries (Post et al. 1991). No other CD46 genes, or CD46-like genes have been identified. As mentioned, CD46 (also known as Membrane Cofactor Protein) protects autologous cells from complement-mediated cytolysis. This complement regulatory protein is a polypeptide of approximately 60 kDa and is composed of numerous repeating units (known as CCP's) containing several N-glycosylation sites, a serine-threonine-proline rich region (STP) containing several O-linked glycosylation sites, a transmembrane region and a cytoplasmic tail. Alternative splicing of CD46 transcripts results in different combinations of different parts of the protein (Liszewski et al. 1991). The N-terminus, identical for all known isoforms, contains three potential N-glycosylation sites in CCP-1, CCP-2 and CCP-4. The STP domain is extensively O-glycosylated. The protein is found to be widely distributed among cell types, including fibroblasts, endothelial cells and epithelial cells in many organs.

There is an apparent discrepancy between the broad expression of the CD46 gene (as detected with conventional murine anti-CD46 antibodies) and the plasma cell-restricted expression of the CD46 epitopes (as detected by the phage display library-derived scFv antibody fragments). This was further investigated by using a panel of glycosylation mutants of the CD46 molecule that were stably expressed in Chinese Hamster Ovary (CHO) cells. In these CD46 mutants, in comparison to the wild type CD46 protein BC1, an asparagine in CCP-1, CCP-2 or CCP-4 was replaced by a glutamine resulting in disruption of the N-glycosylation site, resulting respectively in the mutants NQ1, NQ2 and NQ4. In a fourth mutant construct, the entire STP domain with its O-linked glycosylation sites was deleted (ΔSTP). All constructs, the generation of stable cell lines and the culturing of these cells were described by Liszewski et al. (1998). Binding of the antibods to the CD46 glycosylation mutants expressed on the cell surface of the stable cell lines were monitored by immunofluorescent analysis in combination with an anti myc-tag (9E10) antibody and PE linked goat-anti mouse secondary antibody (Southern Biotechnology Associates).

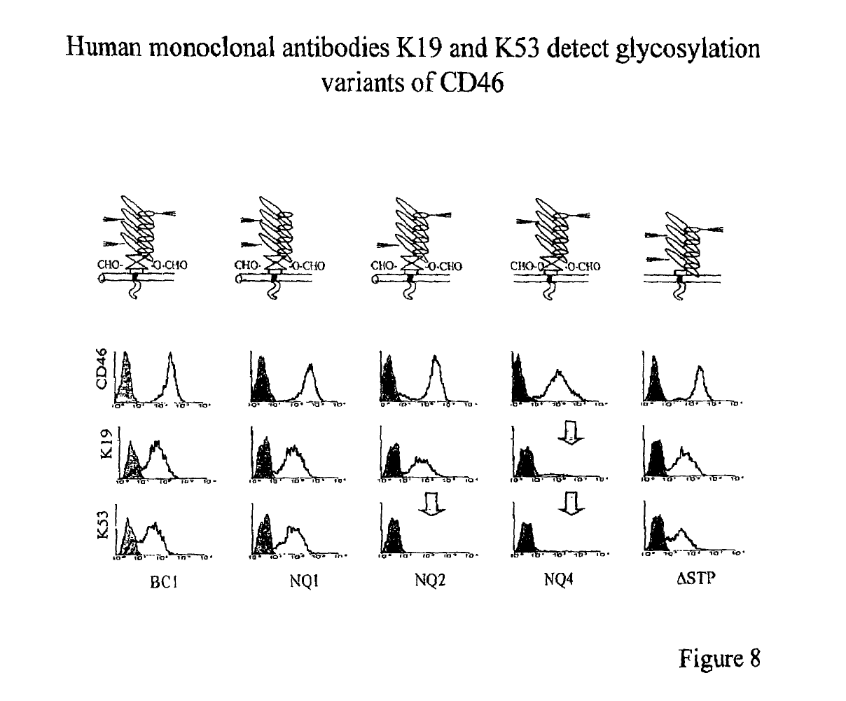

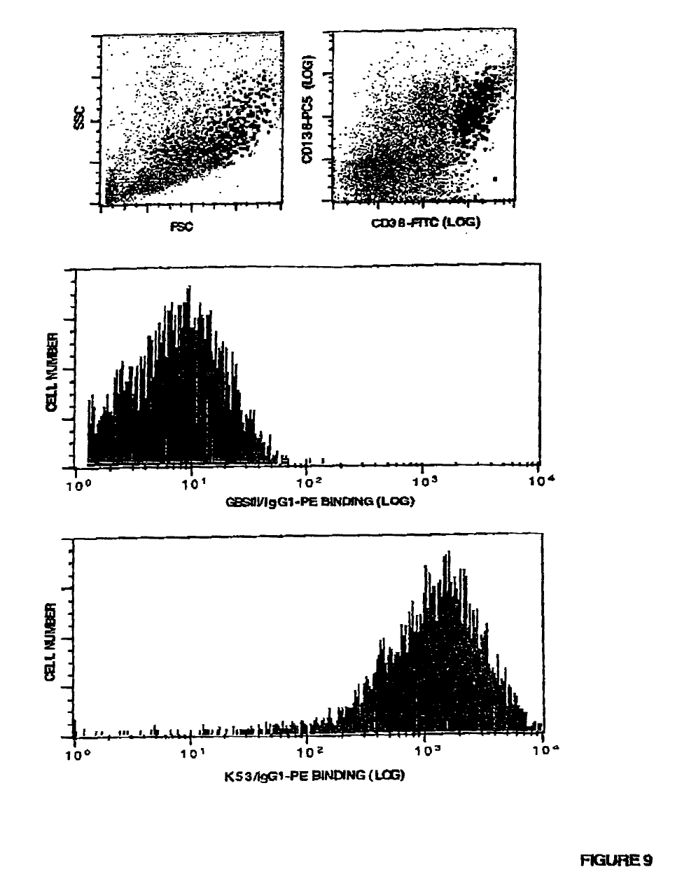

In immunofluorescent analysis, the murine anti-CD46 antibody (J4.48 Immunotech) stained CHO cells transfected with the wildtype CD46 cDNA as well as the transfectants in which each of the glycosylation sites in the CCP domains or the entire STP domain (ΔSTP) were deleted (FIG. 8). In contrast, different staining patterns were obtained with the scFv K19, K29 and K53 antibody fragments. The staining pattern of K19 and K29 were comparable. All three antibodies specifically stained the cell ne transfected with the wildtype CD46 cDNA, as well as the CCP-1 and STP glycosylation mutants (FIG. 8). K19 and K29 also bound to the CCP-2 glycosylation mutant but completely failed to bind to the CCP-4 glycosylation mutant. K53 lost binding to the CCP-2 and CCP-4 glycosylation mutants. Thus, the epitopes on CD46 recognized by K19, K29 and K53 are all dependent on the N-glycosylation of CD46. The involvement of N-glycosylation in binding to CD46 by the human Monoclonal antibodies (huMabs) can also be demonstrated by using the glycosylation inhibitor tunicamycin (see also, Example 9).

### Example 6

Generation of Fully Human IgG1 Monoclonal Anti-CD46 Antibodies From scFv's K19, K29 and K53

The engineering and production of the human IgG1 monoclonal antibodies was described in detail by Boel et al. (2000). Briefly, the VH and VL regions encoding the scFv CD46 antibodies were excised and recloned into vectors for expression of complete human IgG1/k molecules in BHK cells transfected with the furine gene (to yield fur-BHK21 cells) in a two step cloning procedure. The scFv fragments were first cloned in pLEADER (Boel et al. 2000) to add the T cell receptor a chain HAVT20 leader peptide sequence and a splice donor site. In the second cloning step, the scFv containing the leader sequence and donor splice site were subcloned in pNUT-Cγ (ECACC deposited) or pNUT-Cκ (ECACC deposited) expression vectors. Both vectors were co-transfected in fur-BHK cells to generate stable cell lines expressing and secreting human monoclonal antibodies (Huls et al. 1999). For production, cells were cultured in serum free UltraCHO medium (Biowhittaker). After 4 days, the medium was collected and the antibodies were purified using a protein A-sepharose column, using procedures known to persons skilled in the art. The resulting proteins were named K19/IgG1, K29/IgG1 and K53/IgG1 respectively.

K53 scFv antibodies showed the highest affinity for the post-translationally modified variant of CD46, present on diseased cells, most of the studies with full sized monoclonal IgG's were performed with the fully human monoclonal antibody (huMab) K53/IgG1 derived from the K53 scFv fragment. As a general negative control, a fully human antibody directed against group B of streptococcus antigen III was used (GBS III), which was also picked up by phage display and engineered in the same way (Boel et al. 2000).

### Example 7

Specific Binding of CD46 Monoclonal Antibodies to Myeloma and Leukemic Tumors

Bone marrow cells from healthy individuals and from MM patients were screened for K53/IgG1 human monoclonal anytibody (huMab) binding. K53/IgG1 and control GBS III huMabs were labeled with PE or APC (IQ Systems, NL). The bone marrow cells were incubated with the huMabs in the presence of 10% normal human serum (NHS) during 15 min at room temperature and were analyzed by FACS. The K53/IgG1 monoclonal antibody co-stained with the myeloma markers CD38 and CD138 (FIG. 9). To characterize the positive multiple myeloma cells, the tissues were co-stained with several (labeled) mouse monoclonal antibodies such as the ones directed against the myeloma markers CD38 and CD138. Co-staining experiments were performed using bone marrow cells as follows, with dilutions between brackets and huMab representing the different fully human IgG1's recognizing CD46:

(i) CD38-FITC (undiluted)/huMab-PE (1:10)/Topro (live/dead marker);

(ii) CD38-FITC (undiluted)/CD56-PE.Cy5 (1:2)/CD19-PE (undiluted)/huMab-APC (1:10);

(iii) CD38-FITC (undiluted)/huMab-PE (1:10)/CD138-PE.Cy5/CD45-APC (1:20);

(iv) CD38-FITC (undiluted)/CD45-PE (1:10)/CD138.PE.Cy5 (1:2)/huMab-APC (1:10).

Also whole blood was used for screening. The staining protocol for these cells was:

(i) CD3-FITC (1:2)/CD19-PE (1:2)/CD45-PE.Cy5 (1:20)/huMab-APC (1:10);

(ii) CD14-FITC (1:5)/CD33-PE (undiluted)/CD45-PE.Cy5 (1:50)/huMab-APC (1:10).

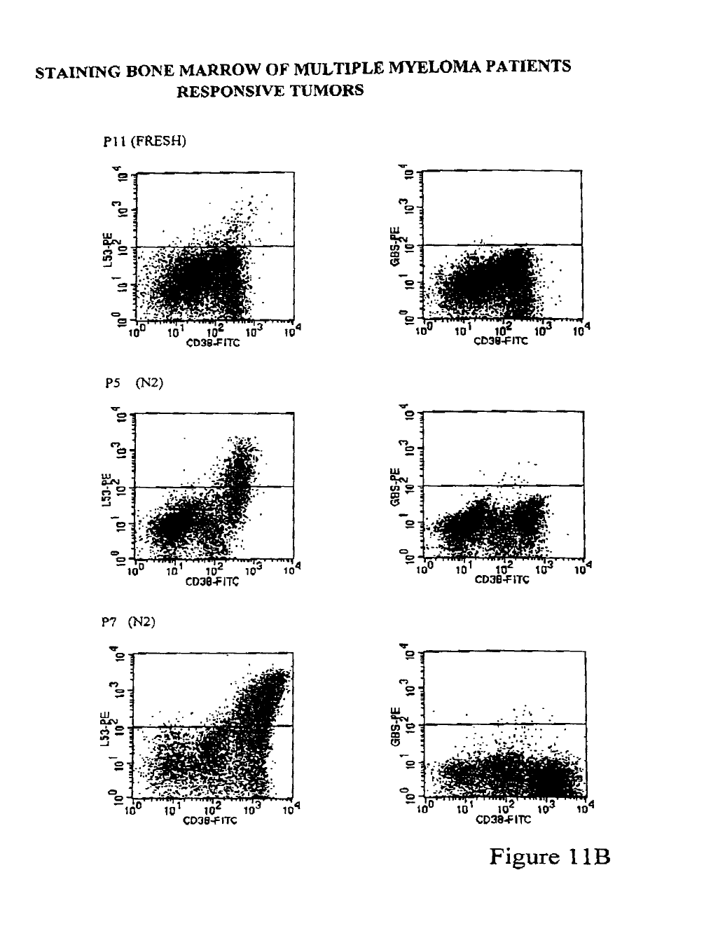

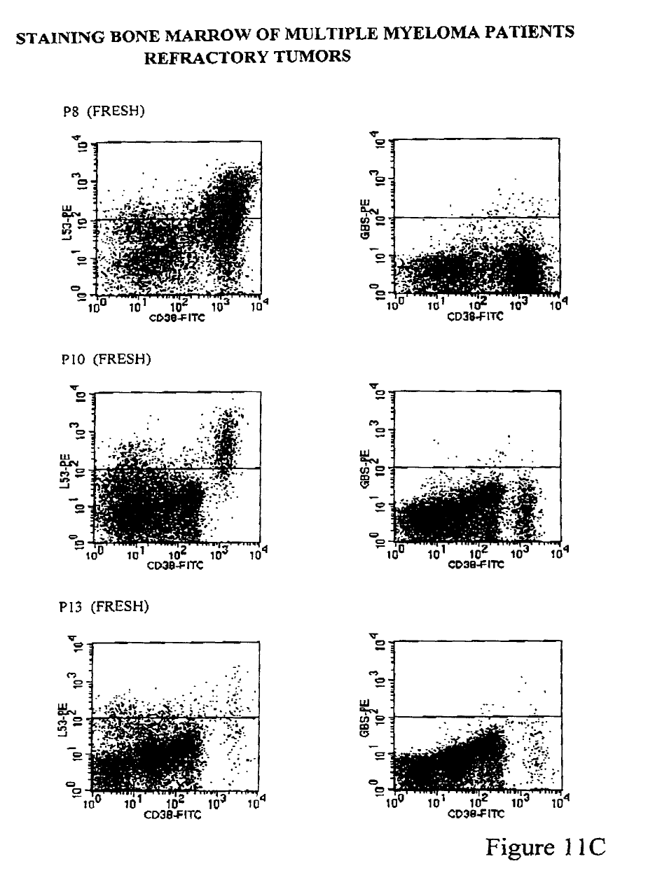

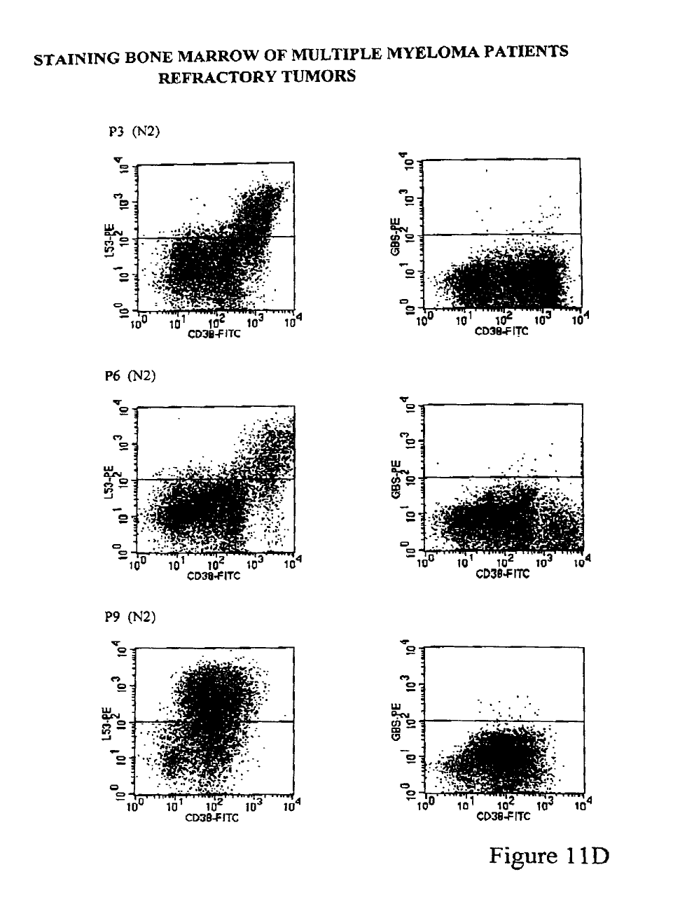

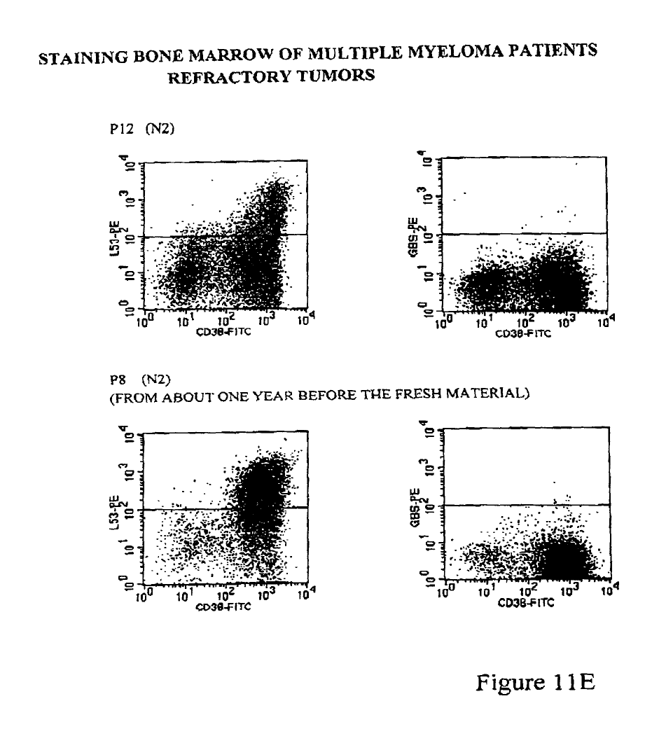

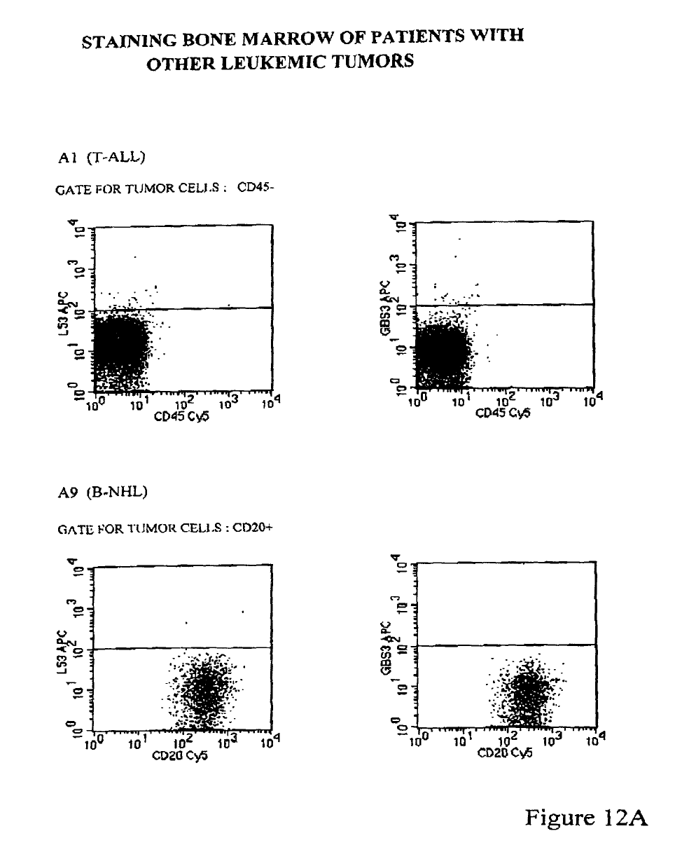

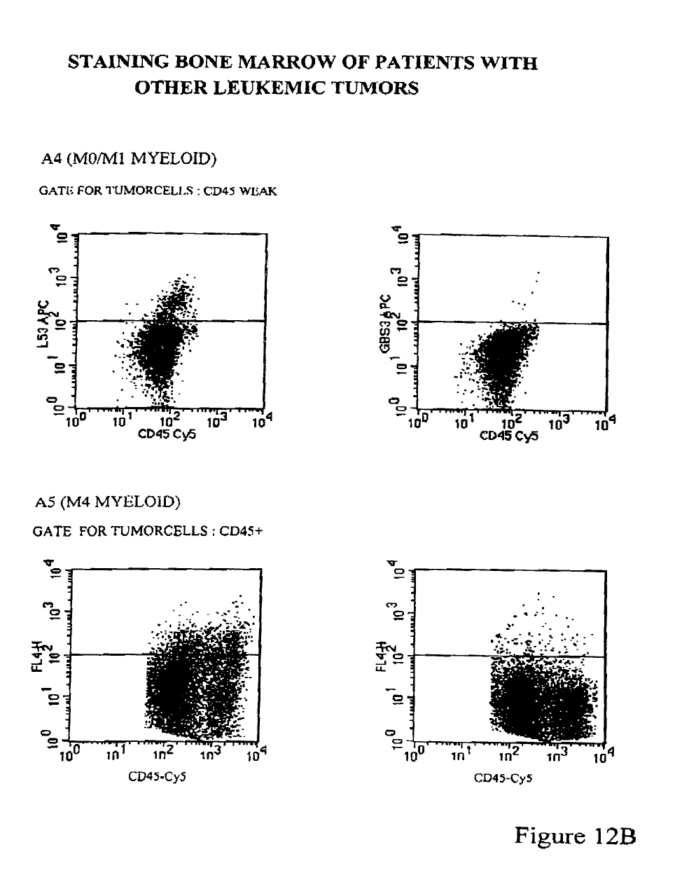

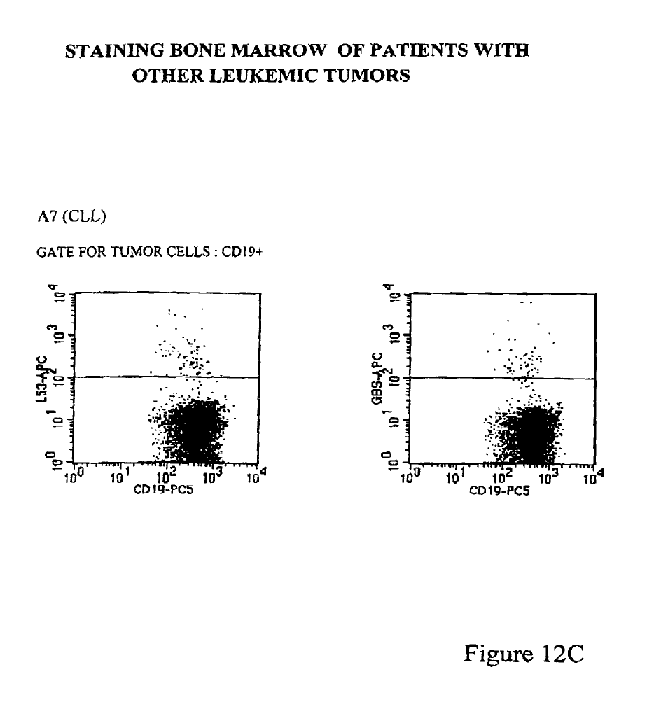

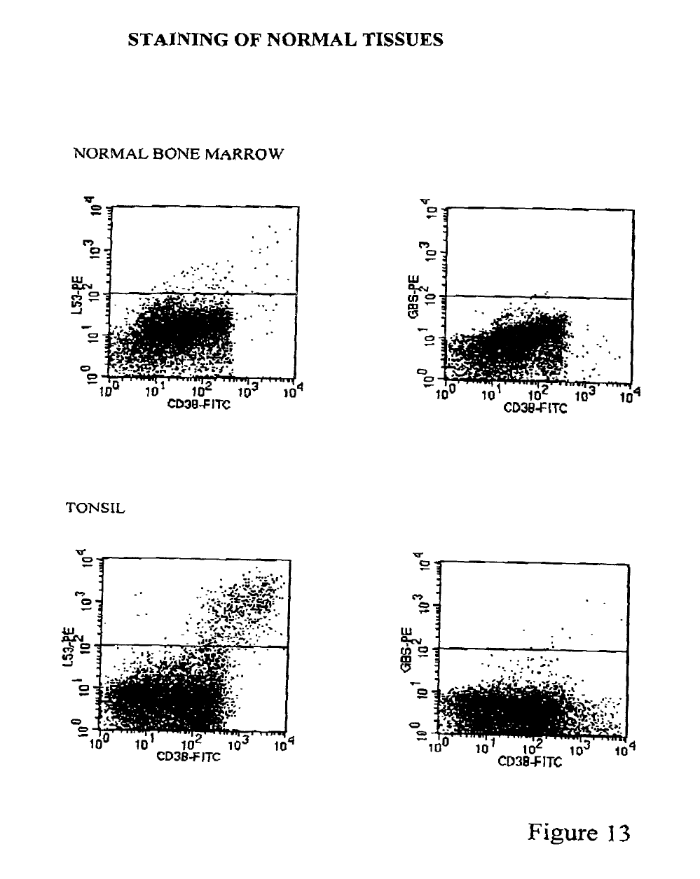

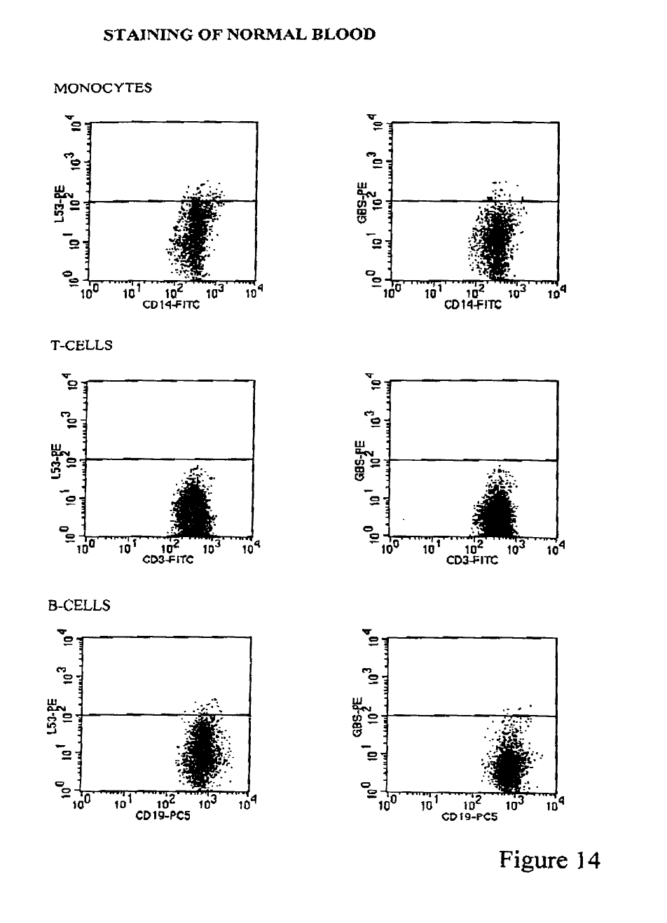

In total, 5×10⁴ cells were measured and analyzed in each experiment, according to general methods known to persons skilled in the art. Table III summarizes the number and status of Multiple Myeloma patient material that was used in the experiments. Table IV summarizes the number and status of patient material with other leukemic tumors that were tested. In FIG. 11 A-E, the dot plots of huMAb K53/IgG1 (here also depicted as L53 on the left side of the specific dot-blots) and GBS III IgG1 control human monoclonal antibodies in relation to CD38 staining are shown of all the patient material tested. All the multiple myeloma cells that were CD38 ^{bright} were highly positive for K53/IgG1 staining. Specific K53/IgG1 binding was observed in primary tumors, as well as in refractory/partly responsive tumors. However, staining of bone marrow of patients with other leukemic tumors, like T-All (T-Acute Lymphatic Leukemia), B-NHL (B-Non Hodgkin Lymphoma's), CLL (Chronic Lymphytic Leukemia), was negative for all the malignancies (FIG. 12A-C). The staining pattern of the K53/IgG 1 antibody in normal bone marrow, normal blood and tonsil was comparable with the K53 scFv fragment (FIG. 13 and 14).

In conclusion, it is clearly shown that among haematopoietic cells the fully human anti-CD46 antibody K53/IgG1, binds to malignant myeloma cells but not to non-malignant cells present in normal bone marrow, tonsils and normal blood. The staining pattern of K53/IgG1, in contrast to CD138 antibodies, was unaffected after storage of tumor material in nitrogen. In contrast, binding of the mouse monoclonal antibody directed against the myeloma marker CD138 with Multiple Myeloma cells is disrupted after freezing with liquid nitrogen, which limits the use of this monoclonal antibody. Therefore, the combination of anti-CD38 monoclonal antibody with the K53/IgG1 is very useful as a diagnostic marker. Binding was not detected in bone marrow cells from healthy individuals and when the control human GBS III antibody was used. Also FACS experiments with whole blood of healthy persons was negative for all the human antibodies used. This negative result was also found with other types of leukemic tumors like Chronic Lymphatic Leukemia (CLL). So, using normal leukocytes and cells from leukemic tumors, the K53/IgG1 clearly binds to Multiple Myeloma cells, but not to non-neoplastic cells.

### Example 8

Binding of Anti-human CD46 Monoclonal Antibodies to Solid Tumors, Cells Derived From Solid Tumors, Myeloma Cells and Leukemic Cells, in Comparison to Normal Tissue

One of the functions of the CD46 protein is to protect the cell from autologous complement attack. One mechanism for tumor cells to deviate the complement attack is by over-expressing the CD46 protein. This over-expression is found in several neoplasia like breast-, cervix-, liver-, colorectal- and gastro-intestinal cancer. The experiments discussed in Example 5 suggest that CD46 cannot only be over-expressed, but also differentially glycosylated. The MM-specific K19, K29 and K53 antibodies apparently distinguish between these different glycosylation forms (FIG. 8). To investigate whether the K19, K29 and K53 antibodies also bind to tumor cells that over-express CD46, the antibodies were incubated with a set of tumor cell lines (MCF-7 (breast), HeLa (cervix), HepG2 (liver) and LS174T (colon, ATCC CL-188) and determined the staining with fluorescent labeled antibodies as described supra. Table I shows the increase in interaction between several solid tumor-derived cell lines with K19, K29 and K53 antibodies, while Table V shows an extensive number of myeloma cell lines, leukemic cell lines and solid tumor cell lines that were tested for binding to K53/IgG1 (determined by FACS analysis) in comparison to a conventional murine anti-CD46 antibody J4.48 as a positive control and GBS III as a negative control. Taken together with the data described in Example 5, these data strongly suggest that these cells not only exhibit an over-expression of the CD46 protein but that the protein also has a different glycosylation pattern as compared to wild type, non-tumor cells. It is concluded that the antibodies interact with CD46 proteins on cells that were derived from solid tumors. The long tumor cell line GLC-1 and the human embryo kidney cell line (HEK293) were negative, whereas most of the colon cell lines (DLD-1, HT29, SW480, LS174T versus HCT116:4 out of 5 tumor cell lines) were positive for staining with K53/IgG1. Among the breast tumor cell lines, T47D and MCF-7 were highly positive, whereas MDA-MB231 was less positive for K53/IgG1 binding. A strong correlation between binding to the diseased form (the tumor-cell related post-translationally modified variant) of CD46 using K53/IgG 1 and murine J4.48 (depicted as CD46 in the right columns) was not found. Taken together, the experiments strongly indicate that K53/IgG1 binds to tumor cells that have a different glycosylation pattern as compared to normal cells. Over-expression of CD46, which is a defense mechanism of several tumor cells, can result in an aberrant glycosylation and an accumulation of high mannose type glycoforms. K53/IgG1 possibly prefers to bind to these abberant glycoforms of CD46, which is most likely a tumor specific epitope of CD46. Hence, the multiple myeloma associated variants are an example of a class of novel epitopes, herein referred to as post-translationally modified molecular markers, or as disease associated molecular markers, which are characterized by their aberrant post-translational modification as compared to their correctly post-translationally modified counterpart. The reason for the occurrence of a post-translational modification in a protein present on a diseased cell or associated with diseased cells (or a diseased state of a species) can be multiple. For instance, the over-expression identified on tumor cells for certain proteins can lead to incorrect post-translational modifications such as misfolding, multimerization and/or aberrant glycosylation and the like. Besides this, it is also possible that such incorrect post-translational modifications, described herein are responsible for, or play a role in the fact that the cell to which they are associated with, are diseased. Either way, these disease associated post-translationally modified proteins can be used as markers for the diseased state of the species and/or the diseased state of the cell to which they are associated, in contrast to their correctly post-translationally modified forms, herein referred to as their counterparts, present on non-diseased cells.

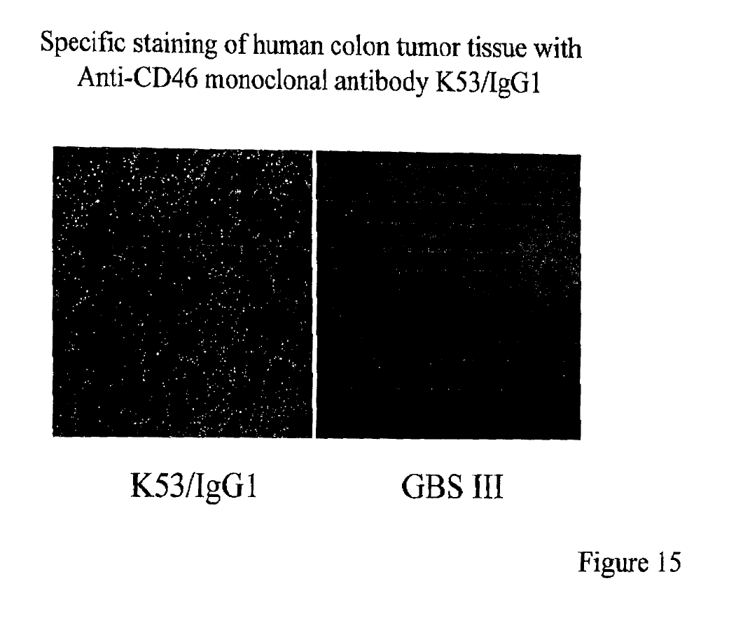

We also tested whether K53/IgG1 interact with CD46 present on cells in solid tumors and not with surrounding tissues. Therefore, tumor material was either obtained directly after surgery and pathology, or from commercial sources and stained with the different anti-human CD46 antibodies. Several tissue sections were derived from Novagen (Germany) and from the University Medical Center (Utrecht, NL). Tissues were fixed in 4% paraformaldehyde, sectioned at 5-6 μm thickness and pre-treated with 10% normal human serum/PBS during 1 h at room temperature. The slides were incubated with PE-linked K53/IgG1 (diluted 1:5) or PE-linked GBS III (diluted 1:5) in 1% BSA/5% NHS/PBS for 1 h at room temperature, washed three times for 10 min with PBS/0.1% Tween, followed by anti-rabbit PE (diluted 1:100) in 1% BSA/5% NHS/PBS (1 h, room temperature), subsequently washed again three times for 10 min and incubated with anti-rabbit IgG-FITC (diluted 1:100) for 1 h at room temperature. After extensive washing, slides were covered with mounting medium and studied using fluorescent microscopy. The results of the immuno histochemistry are shown in Table VI for normal tissues and VII for tumor tissues. None of the normal tissues tested here stained positive for K53/IgG1, whereas K53/IgG1 bound to some human breast and colon tumor sections. One staining experiment on human colon tumor tissue stained positive with K53/IgG1 and GBS III as a negative control was also envisaged by microscopy. The results shown in FIG. 15 indicate that GBS III does not exhibit any significant staining while K53/IgG1 stained the tumor cells to a highly significant level.

### Example 9

Effect of Glycosylation Inhibitors on CD46 Binding

To determine the role of N-glycosylation in K53/IgG1 binding, K53/IgG1 positive tumor cell lines (LS174T cells and MCF7 cells) are treated with the N-glycosylation inhibitor tunicamycin during three days at concentrations of 0.3 and 1 μg/ml. The cells were stained for CD46 using PE-linked fully human monoclonal antibodies K53/IgG1 and negative control antibody GBS III, and commercially available murine anti-CD46 antibody J4.48, which binds to all CD46 derivates (see also FIG. 8). For this purpose, the antibodies were used in combination with a second PE-labeled goat-anti-mouse antibody. The binding capacity was detected and analyzed by Fluorescence-Activated Cell Sorting (FACS) using techniques well known to persons skilled in the art. Treatment with tunicamycin of both cell types resulted in a dramatic loss of K53/IgG1 binding, whereas CD46 expression (as measured by the murine CD46 antibody J4.48) was not diminished. Thus, the total disruption of N-glycosylation by tunicamycin results in a change of K53/IgG1 binding. This shows K53/IgG1 recognizes an epitope that is the result of an aberrant glycosylation, most likely N-glycosylation.

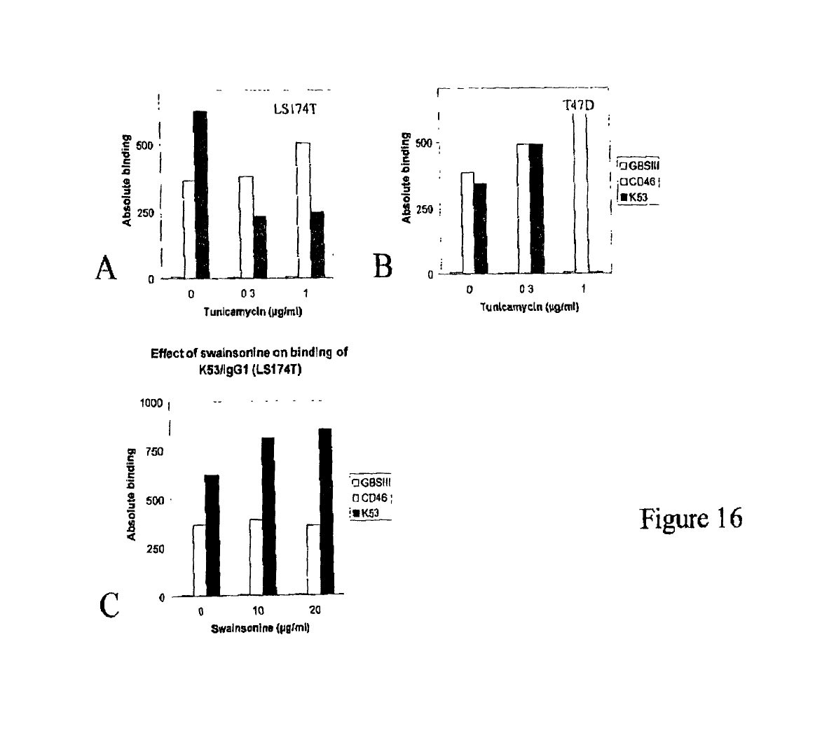

Besides tunicamycin (which prevents the first step of the core oligosaccharide construction), the effect of swainsonine on K53/IgG1 positive tumor cells was also tested. The target enzyme of swansonine is Mannosidase II and therefore this compound prevents the removal of mannose residues of the high mannose structure. Treatment with swainsonine generally results in an accumulation of high mannose type glycoproteins. Using LS 174T colon tumor cells, swainsonine treatment with 20 and 30 μg/ml during 3 days resulted in an significant increase of K53/IgG1 binding, whereas the binding of J4.48 remained similar (FIG. 16)

These data strongly indicate that K53/IgG1 recognizes an aberrant CD46 glycoform and might prefer to bind to a high mannose type of the CD46 protein.

### Example 10

DCC and CDCC Assays, in vitro Killing Assays

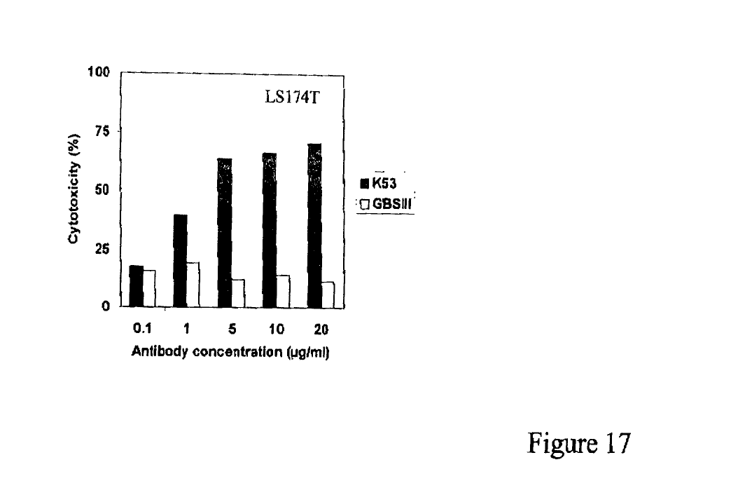

The tumor cell killing activity of the anti-CD46 antibody using human peripheral blood mononuclear cells was evaluated using Multiple Myeloma cells and tumor cell line LS174T derived from colon as target cells in Antibody and Complement Dependent Cellular Cytotoxicity (ADCC and CDCC) assays. The target cells were labeled with 30 μg/ml calcein (Molecular Probes) for 5 min at 37°. After extensive washing, isolated human mononuclear cells were added to the target cells at an effector:target ratio of 40:1. Complement mediated lysis was performed with 50 μl serum in a final volume of 200 μl. Cells were incubated at 37° C. in the presence of various concentrations of K53/IgG1, GBS III negative control antibodies or PBS. After 4 h, propidium iodide was added as a marker for cytotoxicity and cytolysis was determined by FACS analysis. The percentage cellular cytotoxicity was calculated as follows: The percentage specific cytolysis=[number of double positive (propidium-iodide/calcein positive) cells]/total number of calcain positive cells×100. Cytotoxicity was determined in the presence of serum (CDC), in the presence of effector cells (ADCC) and in the presence of both (CDCC). The results of K53/IgG1 and K29/IgG1 are shown in Table II, while the results of K53/IgG1 is also graphically depicted in FIG. 17 and clearly indicate that both human monoclonal antibodies directed against CD46 glycoforms are effective in killing target cells in the presence of blood effector cells.

### Example 11

In vivo Tumor Cell Killing by Anti-CD46 in a Xenograft Model of Colon Carcinoma

LS174T human carcinoma cells were cultured in DMEM supplemented with 10% FCS, penicillin (100 IU/ml) and streptomycin (100 μg/ml). Prior to xenografting, cells were trypsinized, collected, washed twice with PBS and resuspended in PBS (1×10⁶ cells/100 μl). K53/IgG1, the negative control GBS III and the positive control UBS-54, directed against human EpCAM were essentially produced as described by Boel et al. (2000) and as explained in Example 14. Briefly, the V_{H} and V_{L} genes encodig scFv K53, UBS-54 and GBS III were cloned into expression vectors for synthesis of complete IgG1 molecules (Boel et al. 2000). The constructs were then stably expressed in BHK-21 cells transfected with the furine gene (Baby Hamster Kidney cell line fur-BHK21 (BHK-21, ATCC CCL-10). Cells were maintained at 37° C. in a 5% CO₂ humidified incubator in Iscove's modified Dulbecco's medium containing 10% FCS, 2 mM glutamine, penicillin (100 IU/ml) and streptomycin (100 μml). For production, cells were cultured in serum free UltraCHO medium. After 4 days the medium was collected for antibody isolation. One batch of K53/IgG1 was produced in HEK 293 cells (human embryonal kidney cell line 293, German Collection of Microorganisms and cell Cultures, Dept. Human and Animal Cell Cultures ACC 305). The engineering and production of human K53/IgG1 monoclonal antibodies in HEK 293 and PER.C6 cells is described in detail in Example 14.

The antibodies were purified using a protein-A column. The columns were washed with 50 ml PBS, and subsequently supernatant of the monoclonal antibody producing cells was applied to the column. After washing the column with 20 ml PBS, the proteins were eluted with 0.1 M citric acid pH 3.0. Fractions of 1 ml were collected and immediately neutralized with 200 μl 1 M Tris. The protein containing fractions, as determined by spectrophotometry at 280 nm, were pooled and dialyzed extensively against PBS at 4° C. The monoclonal antibodies were filtered (0.20 μm) and final concentrations were determined using the Biorad Protein Assay.

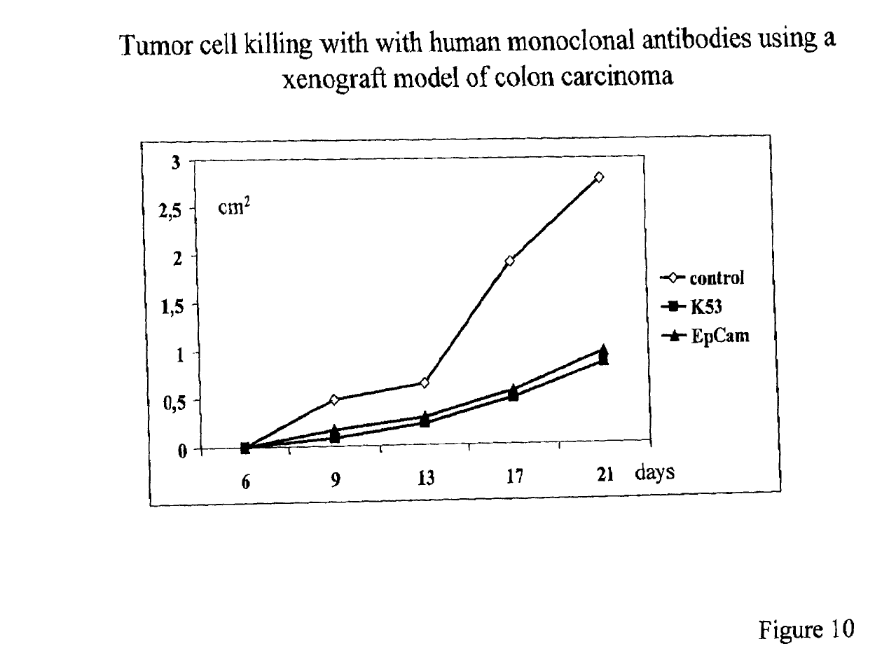

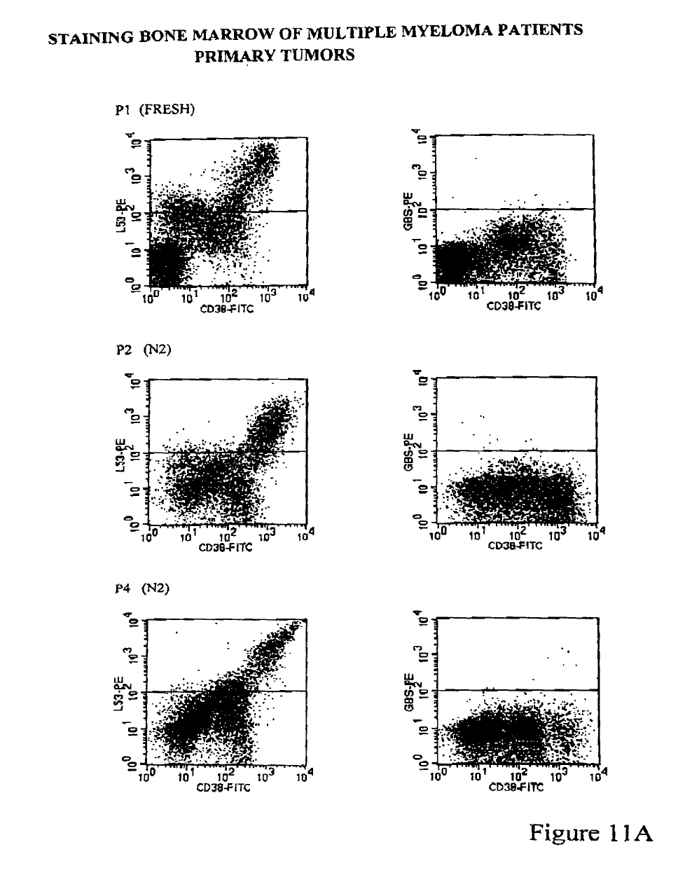

To evaluate the in vivo tumor cell killing capacity of the anti-CD46 monoclonal antibodies, two sets of experiments were performed; in the first set, nine-week old NOD/SCID mice were injected subcutaneously into both flanks with 1×10⁶ LS174T human colon carcinoma cells (day 0). The next day, two groups of 6 animals each were treated: one group with 200 μg K53/IgG1 and the other (control) group with 200 μg GBS III/IgG1 human monoclonal antibodies. On day 3 and 6, the treatment was repeated with 100 μmg monoclonal antibodies. The treatment effects were evaluated by measuring the mean tumor size (maximal length×maximal width) during 3 weeks using methods known to people skilled in the art. Significantly, the tumor growth was markedly retarded by the CD46 human monoclonal antibody (FIG. 10).

In a second set of experiments, seven week-old Balb/c (nu/nu) mice were injected subcutaneously into both flanks with 1×10⁶ LS174T cells (day 0). On day 1 (Group A), day 6 (Group B) and day 9 (Group C), three groups of five animals were treated with 300 μg antibody: one group with K53/IgG1, one with GBS III (negative control) and one with UBS-54 (positive control, Huls et al. 1999). On day 3 and 6 (Group A), 9 and 12 (Group B) and 12 and 15 (Group C) the treatment was repeated with 150 μg antibody. The antibody treatment is summarized in Table VIII. K53/IgG1 produced in BHK-21 cells was used for the antibody treatment on day 1 (group A). A mixture of K53/IgG1 produced in HEK 293 cells and K53/IgG1 produced in BHK-21 cells (90% and 10% respectively) was used for the antibody treatment on day 9 (Group B and C). In all other cases K53/IgG1 produced in HEK 293 cells was used. Treatment effects were evaluated by measuring the mean tumor size (maximal length×maximal height×maximal width) on day 9, 13, 15, 17 (Group A and B) or day 18 (Group C).

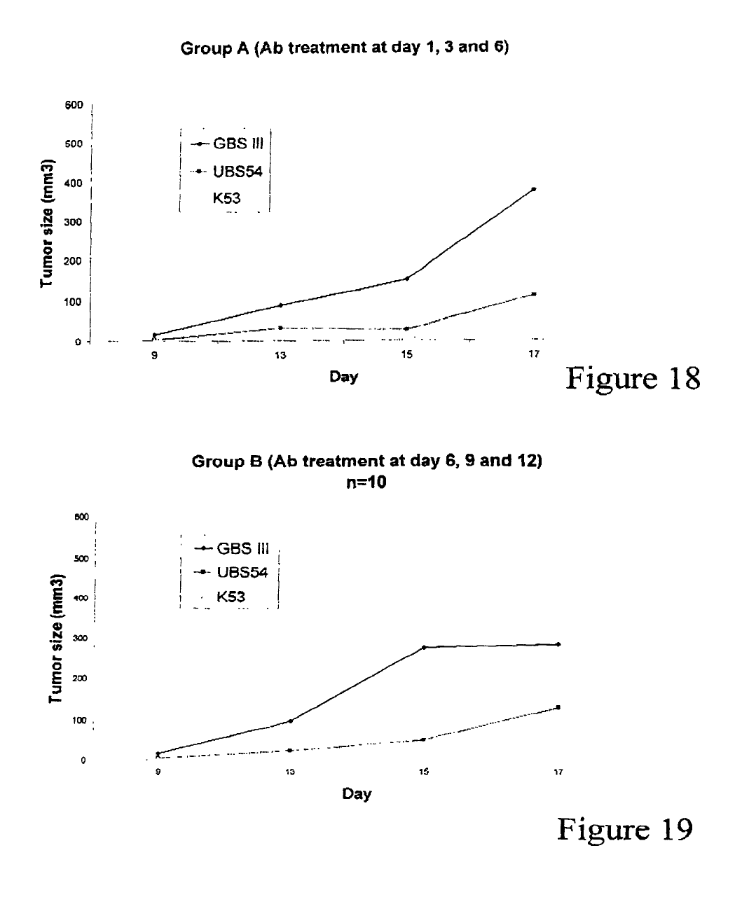

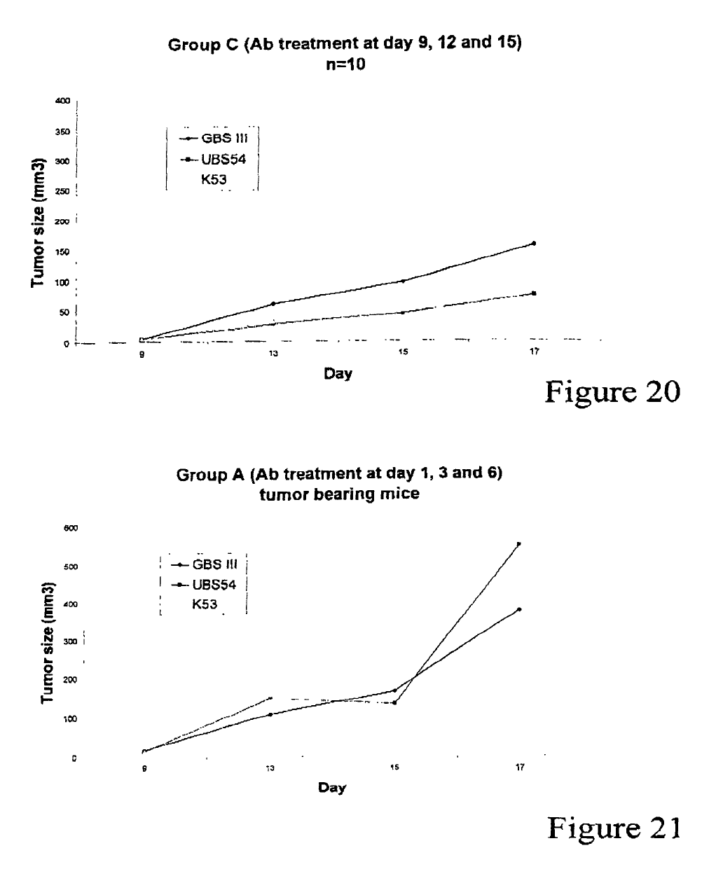

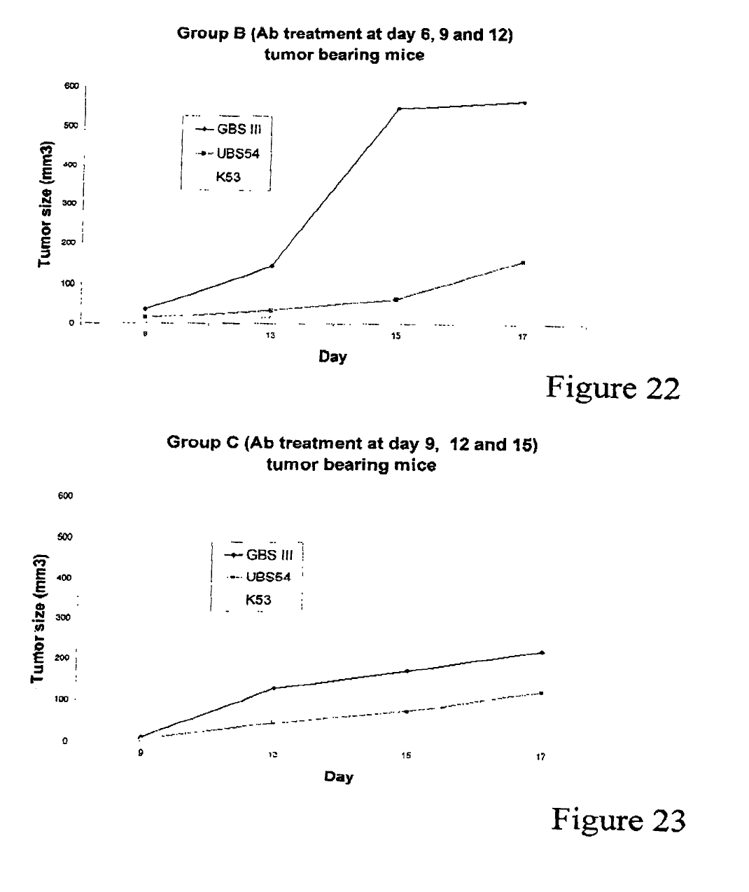

The therapeutic potential of K53/IgG1 was evaluated by measuring the mean tumor size using procedures well known to persons skilled in the art. When the K53/IgG1 antibody treatment started on day 1 (Group A), the tumor growth was significantly retarded when compared to the tumor growth in mice treated with the control antibody GBS III (Table IX, FIG. 18). The effectivity of K53/IgG1 was comparable to the UBS-54 treatment. After 17 days only 3 mice (3 out of 10 inoculation sites) developed a tumor. The mean size of the tumors that did develop was not significantly smaller than the tumors from the control animals (Table X, FIG. 19). Also when the antibody treatment was started at day 6 or 9 (Group B and C. respectively) there was a clear tendency of tumor growth retardation when the animals were treated with K53/IgG1 or UBS-54, compared to the mice treated with the negative control antibody GBS III (Table IX, FIGS. 20 and 21). Although there was no difference in the number of mice that developed a tumor (Table X), the size of the tumors in the K53/IgG1 and UBS-54 treated mice was smaller than the tumors of the GBS III treated mice (Table X, FIGS. 22 and 23).

These results show that when the K53/IgG1 antibody treatment was started immediately (1 day after xenografting) the number of animals developing a tumor is reduced by 70%. Only 3 mice (3 out of 10 inoculation sites) developed a tumor, whereas in the GBS III control group all animals developed a tumor (10 out of 10 inoculation sites). When the antibody treatment was started at later time points, growth retardation of the tumors was also observed. Although the number of animals developing a tumor is not reduced, the mean size of the tumors was 60% to 80% (Group C. and B respectively) smaller in animals treated with K53/IgG1 than in animals treated with the control antibody GBS III.

These results strongly suggest that K53/IgG1 has therapeutic potential, either as such or in a conjugated form.

### Example 12

Determination of Oligosaccharide Content of CD46 Present on Tumor Cells That are Specifically Recognized by K19, K29 and K53

The glycosylation status of the CD46 protein present on normal and cancer related cells, recognized by K19, K29, K53 and other binding molecules is determined. Amongst others, CD46-positive tumor cells (MCF-7, LS174T and MM) and CD46-negative Peripheral Blood Lymphocytes (PBL) are subjected to immunoprecipitation using different CD46 specific monoclonal antibodies and methods known to persons skilled in the art. This is followed by western blot analysis using HRP-linked lectins. These lectins discriminate between different sugar molecules present in the oligosaccharide backbones and are used for western blotting according to methods described by the manufacturers. The lectins can specifically recognize the following molecules: A. High mannose (con A from Canavalia ensiformis), B. Higher branched complex type oligosaccharides (WGA from Triticum vulgare and PHA-L from Phaseolus vulgaris), C. Sialic acids (LFA from Limax flavus) and D. Fucose residue (UEX-I from Ulex europaeus). The glycosylation status of the CD46 protein is determined on sets of tumor cells and compared to the status of the oligosaccharides present on CD46 which is expressed on the surface of normal cells or which is soluble and present in bodily fluids in healthy individuals and cancer patients. Moreover, CD46 glycosylation is detected also within one tumor type and/or one tumor derived cell line.

These experiments are followed by procedures to find which type(s) of glycosylation is responsible for binding to which binding molecule, more in particular to the antibodies K19, K29 and K53 and what glycosylation patterns and sugar backbones are overlapping. These procedures include polysaccharide analysis with NMR and mass spectrometry using procedures well known in the art. Subsequently, after determining what glycoforms of the CD46 protein are present on what tumor cells, the correct monoclonal antibody will be used to induce complement activation by the best interacting binding molecule in in vitro and in in vivo studies.

### Example 13

Specification of Amino Acid Residues in the Variable Domain of the Antibodies That Determine the Binding to Different CD46 Glycoforms

The residues in the variable domains in the antibodies that are selected using the methods described supra are responsible for the specific interaction between the CD46 protein and the recombinant IgG1. Moreover, they specify the interaction with the different glycosylation patterns that are present on the protein expressed on the surface of Multiple Myeloma cells and other cells derived from solid tumors. The responsible amino acid residues are determined by randomly altering the amino acid order present in CDR3 and other variable regions. Subsequently, the mutagenized regions are incorporated into full IgG1 molecules, expressed, purified and used in binding assays with MM and other tumor derived cells. Positive binding molecules are subsequently selected.

### Example 14

Instruction of Expression Vectors and Production of the Recombinant Fully Human Anti-CD46 Monoclonal Antibody K53/IgG1 in Human PER.C6 and HEK 293 Cells

It is possible that recombinant monoclonal antibodies that are selected by the methods described supra, do render immune responses in humans that are being treated with these binding molecules due to the fact that there might be unrelated non-human post translational modifications present on these therapeutic molecules. For this it is preferred to produce these fully human monoclonal antibodies in a system that comes closest to the human situation. Therefore, heavy and light chains of the fully human monoclonal antibody K53/IgG1 were cloned in several eukaryotic expression vectors that are described, amongst others, by Huls et al. (999) and in WO 00/63403.

The final expression vector(s) are subsequently transfected into human PER.C6 cells (ECACC deposit 96022940) using transfection procedures well known to persons skilled in the art. Then, cells that have stably integrated versions of both heavy and light chain are selected by double selection with G418 (GIBCO) and/or Hygromycin (GIBCO). Another procedure involves subsequent selection in which first Hygromycin is added to the medium in which the cells are growing and outgrowing colonies are further selected with the addition of G418 to the medium or vice versa. In other systems, both heavy and light chain are under the same selection pressure because they were cloned in one expression vector or they are cloned in similar expression vectors carrying the same selection marker. The method of using one expression vector carrying both the heavy and light chain in combination with the neomycin selection marker is described below in more detail.

In yet another method, a Methotrexate-DHFR slection method (Urlaub et al. 1983) is applied in which it is possible to amplify the integrated plasmids and thereby increasing the production levels of the recombinant antibodies. These methods have been applied by others by making use of for instance a hamster cell line, CHO that was deficient for its endogenous DHFR. Positive cell clones are picked and subcultured according to methods known to persons skilled in the art. Then specific production rates are determined and the best clones are selected for further outgrowth, banking, stability of expression, amplification, suspension growth and optimal growth versus production in large bioreactors. The recombinant fully human monoclonal antibodies directed against the differentially glycosylated CD46 proteins are purified from the supernatant using methods well known to persons skilled in the art and used in specificity experiments, ADCC and CDCC assays and in vivo tumor-killing experiments. These fully human monoclonal antibodies produced in human cells are furthermore used in specificity- and pharmacokinetics studies and half-life experiments.

Cloning of K53/IgG1 Mammalian Expression Vector pCD46-3000/Neo

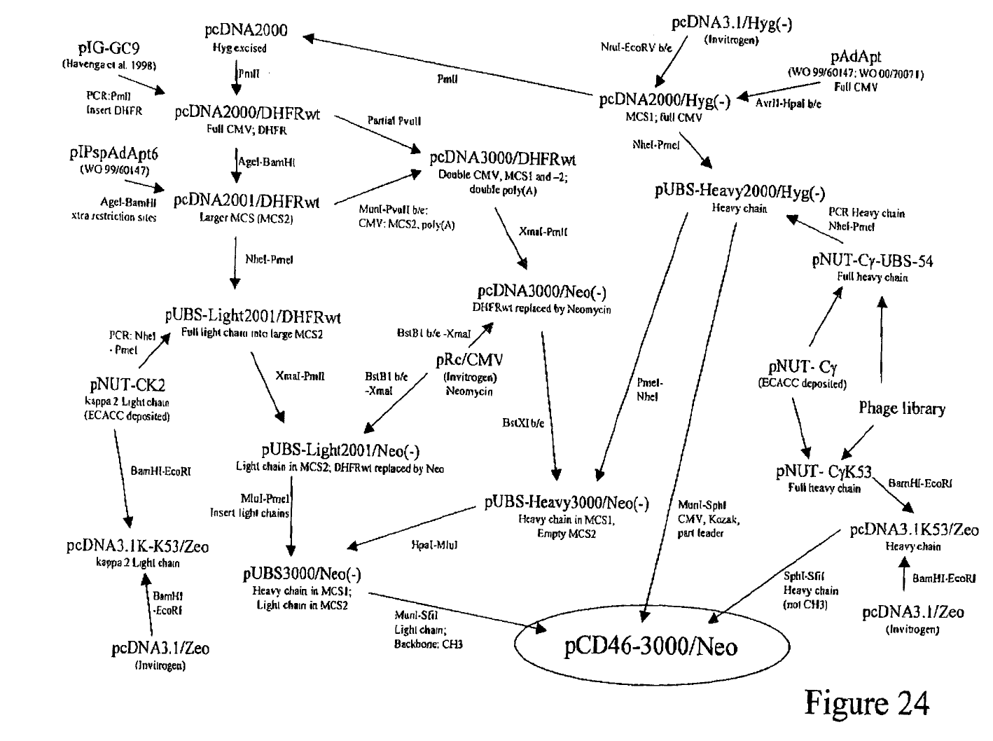

The cloning procedures given below were performed to construct the K53/IgG1 expression vector that was used to transfect mammalian cells and to obtain stable cell lines. An overview of all cloning steps is depicted in FIG. 24.

In order to construct a plasmid containing both the heavy and the light chain of the fully human anti-CD46 monoclonal antibody K53/IgG1, first a backbone had to be constructed. In contrast to the commercially available vectors this newly formed backbone contains: two CMV promoters, two Multiple Cloning Sites (MCS) and two Bovine Growth Hormone (BGH) poly-adenylation (poly(A)) sites. This was achieved by combining the described regions of two vectors. After establishment of the backbone, the light and heavy chains were cloned into the vector. Generally, a human IgG1 consists of heavy and light chains which both contain variable and constant domains. The variable domain determines the specificity of the antibody, while the constant domain is preserved.

pcDNA3.1/Hyg(−) (Invitrogen) was digested with NruI and EcoRV, dephosphorylated at the 5′ termnini by temperature sensitive Shrimp Alkaline Phosphatase (tSAP, GIBCO Life Science Techn.) and the plasmid fragment lacking the immediate early enhancer and promoter from cytomegalovirus (CMV) was purified from agarose gel using a GeneClean kit (Bio 101, Quantum Biotechnologies Inc.). pAdApt(WO 99/60147 and WO 00/70071) containing the full length CMV enhancer/promoter (−735 to +95) next to overlapping Adenovirus serotype 5-derived sequences to produce recombinant Adenovirus, was digested with AvrII filled in with Klenow polymerase (GIBCO) and digested with HpaI. The fragment containing the CMV enhancer/promoter was isolated from agarose gel and ligated blunt/blunt into the NruI/EcoRV fragment of pcDNA3.1/Hyg(−). This destroys the HpaI, EcoRV, NruI and AvrII sites. The ligation product was transformed to competent DH5α cells (GIBCO) and plated on LB/AMP plates. Thirty colonies were picked and cultured in LB/AMP medium for plasmid DNA isolation (according to the Qiagen miniprep procedure). The plasmid DNA of the thirty clones was controlled by restriction enzyme analysis using the restriction enzyme HincII. Eight clones turned out to contain the correct plasmid. One clone was used for further experiments. The resulting plasmid was designated pcDNA200/Hyg(−).

pNUT-Cγ (ECACC deposited) comprises the constant domains, introns and hinge region of the human IgG1 heavy chain (Huls et al. 1999). A synthetic intron and the variable domain of the gamma chain from the fully humanized monoclonal antibody UBS-54 was introduced upstream of the first constant domain in this plasmid resulting in pNUT-Cγ-UBS-54 essentially as described by Boel et al. (2000). The variable domain herein is preceded by the following leader peptide sequence:

| | | |
|---|---|---|
| peptide sequence: | MACPGFLWALVISTCLEFSM | (SEQ ID NO:_) |
| (DNA sequence: | 5′-ATG GCA TGC CCT GGC TTC CTG TGG GCA CTT | (SEQ ID NO:_). |
| | GTG ATC TCC ACC TGT CTT GAA TTT TCC ATG-3′) | |

pUBS-Heavy2000/Hvg(−) was generated in order to insert the Kozak sequence before the leader sequence of the heavy chain and place the sequence encoding the UBS-54 heavy chain under a CMV promoter. The entire gamma chain from UBS-54 was amplified from pNUT-Cγ-UBS-54 by touchdown PCR using the upstream primer UBS-UP and the downstream primer CAMH-DOWN. The sequence of UBS-UP is as follows: 5′-GAT C AC GCG TGC TAG CCA CCA TGG CAT GCC CTG GCT TC-3′ (SEQ ID NO:______) in which the introduced MluI (ACGCGT) and NheI (GCTAGC) sites are underlined and the perfect Kozak sequence is italicized. The sequence of CAMH-DOWN is as follows: 5′-GAT CGT TTA AAC TCA TTT ACC CGG AGA CAG-3′ (SEQ ID NO:______) in which the PmeI recognition site is underlined. The resulting PCR product was digested with NheI and PmeI restriction enzymes. The DNA fragment was purified over agarose gel using GeneClean and ligated to pcDNA2000/Hyg(−) digested with NheI and PmeI, dephosphorylated with tSAP and purified over gel using GeneClean. The ligation product was transformed to competent DH5α cells and plated on LB/AMP plates. Eight colonies were picked and cultured in LB/AMP medium for plasmid DNA isolation. The plasmid DNA of the clones was controlled by restriction enzyme analysis using NcoI. Six clones displayed the correct digestion pattern thereby confirming they contained the correct plasmid. One clone was stored as glycerol stock and used for further plasmid isolation. The resulting plasmid was named pUBS-Heavy2000/Hyg(−).

Instead of hygromycin, other selection markers were to be used in further constructs. For this, a plasmid was generated lacking a selection marker. pcDNA2000/Hvg(−) was digested with PmII, and the 4.7 kb plasmid lacking the Hygromycin resistance marker gene was purified from agarose gel using GeneClean and subsequently religated. The ligation mixture was transformed to competent DH5α cells and plated on LB/AMP plates. Four colonies were picked and cultured in LB/AMP medium for plasmid DNA isolation. The plasmid DNA of the clones was controlled by restriction enzyme analysis using the restriction enzyme DdeI. All clones turned out to contain the correct DNA plasmid. One clone was used for further plasmid isolation. The resulting plasmid was designated pcDNA2000 that was used to introduce the sequence of another selection marker: dehydrofolate reductase (DHFR). For this, pIG-GC9 (Havenga et al. 1998), containing the wild type human DHFR cDNA was used to obtain the wild type DHFR-gene by polymerase chain reaction (PCR) with non-coding PmII sites upstream and downstream of the cDNA. The primers were: DHFR up:

| | | |
|---|---|---|
| DHFR up: | 5′-GAT CCA CGT GAG ATC TCC ACC ATG GTT GGT TCG CTA AAC TG-3′ | (SEQ ID NO:_) and |
| DHFR down: | 5′-GAT CCA CGT GAG ATC TTT AAT CAT TCT TCT CAT ATA C-3′ | (SEQ ID NO:_). |

In both primers the PmII restriction sites are underlined. After purification (PCR purification kit, Qiagen) the PCR-product was digested with PmII. The fragment was used for ligation into pcDNA2000 digested with PmII, dephosphorylated by tSAP and purified from agarose gel using GeneClean. The ligation mixture was transformed to competent DH5α cells and 15 colonies were picked and cultured in LB/AMP medium for plasmid isolation. The plasmid DNA of the clones was controlled by restriction enzyme analysis using the restriction enzyme DdeI. One clone contained the correct plasmid. This plasmid was named pcDNA2000/DHFRwt.

pcDNA2000/DHFRwt contains a MCS that has unique restriction sites. As this MCS does not contain the specific sites needed to sub-clone the full light chain of a human IgG1, another MCS was introduced. pIPspAdapt 6 (Galapagos Genomics NV; WO 99/60147) was digested with AgeI and BamHI. The resulting MCS fragment has the following sequence:

| | |
|---|---|
| 5′-ACC GGT GAA TTC GGC GCG CCG TCG ACG ATA TCG ATC GGA CCG | (SEQ ID NO:_). |
| ACG CGT TCG CGA GCG GCC GCA ATT CGC TAG CGT TAA CGG ATC C-3′ | |

AgeI and BamHI sites are underlined. This fragment contains several unique restriction enzyme recognition sites and was purified over agarose gel using GeneClean and subsequently ligated to an AgeI/BamHI digested and agarose gel purified pcDNA2000/DHFRwt. This resulted in pcDNA2001/DHFRwt.

pNUT-Cκ2 (ECACC deposited) contains the variable and constant domain of the light chain of human IgG1 kappa 2 (Huls et al. 1999). The light chain of UBS-54 and K53/IgG1 were both of the kappa 2 type and therefore identical. The leader peptide sequence present is the same as the one in pNUT-Cγ described above. pUBS-Light2001/DHFRwt was created from pNUT-Cκ2 and pcDNA2001/DHFRwt in order to obtain the light chain of UBS-54 preceded by the Kozak sequence and under control of a CMV promoter/enhancer. The entire (UBS-54 and K53) light chain of pNUT-Cκ2 was amplified by touchdown PCR using the upstream primer UBS-UP and the downstream primer CAML-DOWN to modify the translation start site. The sequence of CAML-DOWN is as follows: 5′-GAT CGT TTA AAC CTA ACA CTC TCC CCT GTT G-3′ (SEQ ID NO:______). The PmeI restriction site is underlined. After purification the resulting PCR product was digested with NheI and Pmel restriction enzymes and purified over agarose gel using GeneClean. The fragment was ligated to pcDNA2001/DHFRwt digested with NheI and Pmel, treated with tSAP and purified over agarose gel using GeneClean. The ligation mixture was transformed to competent DH5α cells and eight colonies were picked and cultured in LB/AMP medium for plasmid isolation. The DNA of the clones was controlled by restriction enzyme analysis using the restriction enzyme NcoI. Four clones displayed the correct restriction pattern, thereby confirming they contained the correct DNA. One clone was used to generate DNA for further experiments. The resulting plasmid was named pUBS-Light2001/DHFRwt.

Instead of using DHFR or Hygromycin as a selection marker, the Neomycin selection marker was selected for the generation of stable cell lines. Therefore pUBS-Light2001/DHFRwt was used to generate a plasmid containing a Neomycin marker by the exchange of the selection marker sequences. pRc/CMV (Invitrogen) was digested with BstBI, blunted with Klenow and subsequently digested with XmaI. The 840 bp Neomycin resistance gene-containing fragment was purified from agarose gel using GeneClean. The fragment was ligated to pUBS-Light2001/DHFRwt digested with XmaI and PmII restriction enzymes, followed by treatment with tSAP and purification over gel using GeneClean to remove the DHFR cDNA. The ligation of the PmII end and the blunted BstBI site destroyed both restriction recognition patterns. The ligation mixture was transformed to competent DH5α cells and fifteen colonies were picked and cultured in LB/AMP medium for plasmid isolation. The plasmid DNA of the clones was controlled using the restriction enzymes NaeI and SphI. The restriction enzyme analysis confirmed that all of the fifteen picked clones contained the correct DNA. One clone was used to generate DNA for further experiments. The resulting plasmid was named pUBSLight2001/Neo(−)

pcDNA3000/DHFRwt was created by the combination of pcDNA2000/DFHRwt and pcDNA2001/DHFRwt. For this, the new vector would contain a double CMV promoter, a double MCS and a double BGH poly(A). pcDNA2000/DHFRwt was partially digested with restriction enzyme PvuII. There are two PvuII sites present in this plasmid and cloning was performed into the site between the SV40 poly(A) and ColE1, not into the PvuII site downstream of the BGH poly(A). A single site digested mixture of plasmid was dephosphorylated with tSAP and purified over agarose gel using GeneClean. pcDNA2001/DHFRwt was digested with MunI and PvuII restriction enzymes and filled in with Klenow to have both ends blunted. The resulting CMV promoter-linker-BGH poly(A)-containing fragment of 1269 bp was isolated over agarose gel using GeneClean and ligated into the partially digested and dephosphorylated pcDNA2000/DHFRwt. Due to the ligation the PvuII and MunI restriction recognition sites downstream of the SV40 poly(A) sites were destroyed. The ligation mixture was transformed to competent DH5α cells and thirty colonies were picked and cultured in LB/AMP medium for plasmid isolation. The plasmid DNA of the clones was controlled by restriction enzyme analysis using HincII. Six of the picked clones were containing the insert in the correct orientation. One positive clone was used to generate DNA for further experiments. The created plasmid was called pcDNA3000/DHFRwt.

Now, pcDNA3000/Neo(−) was generated by the exchange of the selection marker sequences. It was generated because, as mentioned, the selection marker Neomycin was preferred. pRc/CMV was digested with BstBI, blunted with Klenow and subsequently digested with XmaI. The Neomycin resistant gene-containing fragment was isolated over agarose gel using GeneClean. The isolated fragment was ligated in pcDNA3000/DHFRwt digested with XmaI and PmII, dephosphorylated with tSAP and gel purified using GeneClean. Due to the ligation both the restriction recognition sites of BstBI and PmII were destroyed. The ligation mixture was transformed to competent DH5α cells and 10 colonies were picked and cultured in LB/AMP medium for plasmid DNA isolation. The plasmid DNA was controlled by restriction enzyme analysis using NaeI and PstI/BsmI. Nine out of ten picked clones contained the correct DNA. A positive clone was used to generate DNA for further experiments. The generated vector was named pcDNA3000/Neo(−). Into this backbone plasmid the heavy and light encoding sequences of the UBS-54 anti-EpCAM antibody were inserted.

The next section describes how first the heavy chain was sub-cloned into the vector. The source used of the heavy chain was pUBS-Heavy2000/Hyg(−) since in this plasmid the heavy chain is preceded by the Kozak sequence. pUBS-Heavy2000/Hyg(−) was digested with first PmeI and subsequently with Nhel. The fragment containing the complete heavy chain including Kozak sequence and leader peptide was isolated from agarose gel using Geneclean. The fragment was ligated in pcDNA3000/Neo(−) digested with BstXI, blunted with T4 DNA polymerase and subsequently purified over agarose gel using GeneClean. Due to the ligation both the restriction recognition sites of NheI and PmeI were lost. The ligation mixture was transformed to competent DH5α cells and thirty colonies were picked and cultured in LB/AMP medium for plasmid DNA isolation. The plasmid DNA was controlled by restriction enzyme analysis using KpnI as well as BglII/PstI and AgeI. Among the 30 colonies, 3 turned out to contain the correct plasmid. One of these was used to generate DNA for further experiments. The generated plasmid was named pUBS-Heavy3000/Neo(−).

In order to generate pUBS3000/Neo(−) containing both the heavy and the light chain of UBS-54, pUBS-Heavy3000/Neo(−) was used together with pUBS-Light2001/Neo(−). The latter was used as source of the light chain since in this construct the sequence of the kappa chain was preceded by the Kozak sequence. pUBS-Light2001/Neo(−) was digested with PmeI and MluI. The fragment containing the complete light chain including Kozak sequence and leader peptide was isolated from agarose gel using GeneClean. After isolation, the fragment was ligated in pUBS-Heavy3000/Neo(−) that was digested with HpaI and MluI, gel purified using GeneClean and dephosphorylated with tSAP. Due to the ligation the recognition sites of both HpaI and PmeI were destroyed. The ligation mixture was transformed to competent DH5α cells and 30 colonies were picked and cultured in LB/Amp medium for plasmid DNA was isolation. The plasmid DNA was controlled by restriction enzyme analysis with KpnI as well as with NaeI. With exception of one clone, all clones contained the correct plasmid. One positive clone was used to generate DNA for further experiments. The resulting plasmid was named pUBS3000/Neo(−).

Upstream of the first constant domain pNUT-Cγ received the variable domain of the gamma chain from the fully humanized monoclonal antibody K53/IgG1 that is preceded by a leader peptide essentially according to procedures described by Boel et al. (2000). The leader peptide was identical to the one described above. This resulted in an insert of approximately 2 kb. The generated plasmid was named pNUT-CγK53. This plasmid contains a methallothionine promoter (MT-4 promoter). As this promoter is not ideal for high expression in eukaryotic cells, the heavy chain of K53/IgG1 was subcloned into pcDNA3.1/Zeo (Invitrogen). pNUT-CγK53 was digested with the restriction enzymes BamHI and EcoRI. The fragment containing the complete heavy chain including the proceeding leader sequence was purified and ligated in pcDNA3.1/Zeo digested with BamnHI and EcoRI. The resulting plasmid was named pcDNA3.1K53/Zeo.

Separate from the expression vectors described above, the kappa 2 light chain was also ligated (FIG. 24) into pcDNA3.1/Zeo (Invitrogen) to serve as expression vector in HEK 293 cells (see below). pNUT-Cκ2 was digested with BamHI and EcoRI restriction enzymes. The resulting 1.2 kb fragment was purified over agarose gel using QiaexII gel Extraction kit (Qiagen) and ligated into the 5.0 kb linearized pcDNA3.1/Zeo digested with BamHI and EcoRI restriction enzymes, purified over agarose gel. The ligation mixture was transformed to competent E. coli cells. Then, plasmids of 4 generated clones were checked on correct inserts by restriction digestion with BamHI and EcoRI enzymes. One positive clone was used for further experiments. The correct plasmid was named pcDNA3.1κ2-K53/Zeo.

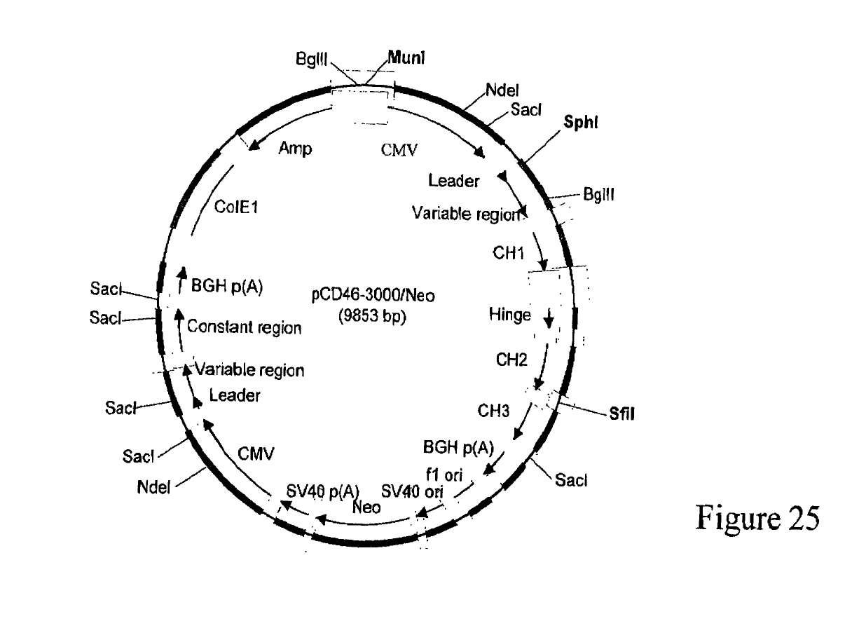

The final construct (pCD46-3000/Neo) that would contain both the kappa 2 light chain and the heavy chain of K53/IgG1 was generated by the exchange of the variable domain of the heavy chain in pUBS3000/Neo(−) with the variable domain of the heavy chain fragment of K53. The heavy chain of K53/IgG1 did not contain the Kozak sequence in pcDNA3.1K53/Zeo, so the 5′ restriction site of the inserted variable domain had to be located within the leader sequence. Due to the fact that there was no unique restriction site present within the leader sequence, a three-point ligation was used to generate the construct pCD46-3000/Neo. For this pcDNA3.1K53/Zeo was digested with SphI and SfiI. With the SphI restriction site situated in the leader sequence and the SfiI in the intron following the CH2 domain a 1759 bp heavy chain fragment was isolated from agarose gel using GeneClean containing: part of the leader sequence+variable domain+intron+CH1+intron+Hinge+intron+CH2. pUBS-Heavy2000/Hyg(−) was digested with MunI and SphI. Hereby a 922 bp fragment containing the sequence of the CMV promoter followed by the Kozak sequence and partly the leader sequence was obtained and isolated from agarose gel using GeneClean. pUBS3000/Neo(−), providing the backbone and the kappa 2 light chain was digested with the restriction enzymes MunI and SfiI. The backbone fragment of 7172 bp was isolated from agarose gel using GeneClean and subsequently de-phosphorylated with tSAP. A three-point ligation with the isolated fragments described above was performed. The ligation mixture was transformed to competent DH5α cells and thirty colonies were picked and cultured on LB/AMP medium for plasmid DNA isolation. The plasmid DNA was controlled by restriction enzyme analysis with BglII/NdeI and BglII/SacI. Of the 30 colonies 17 clones turned out to contain the correct DNA. One of these was used to generate DNA for further experiments. The resulting plasmid was named pCD46-3000/Neo and is depicted in FIG. 25. In conclusion, the region from MunI to SphI is derived from pUBS-Heavy2000Hyg(−) and contains one CMV promoter; the region from SphI to SfiI is derived from plasmid pcDNA3.1K53/Zeo and contains the variable heavy chain and the first two heavy constant domains; the region SfiI to MunI is derived from pUBS3000/Neo(−) and contains the final heavy constant domain and poly(A) sites, resistance markers, plasmid replication sequences and the complete kappa 2 encoding region.

Selection of HEK 293 and PER.C6 Cells Expressing the Fully Human Anti-CD46 Antibody K53/IgG1

PER.C6 cells (ECACC deposit 96022940) were transfected with pCD46-3000/Neo (construct described above, FIG. 24 and 25). This plasmid expresses both light and heavy chains of the K53/IgG1 molecule, and also encodes a neomycin resistance marker (Neo). Cells were grown in DMEM supplemented with serum in the presence of Geneticin (G418) to select PER.C6 clones that were stably transfected for this plasmid. For the transfection, PER.C6 cells (passage number #41) were seeded in 10 cm tissue culture dishes in DMEM (Gibco) supplemented with 10% FBS (Gibco) and 1% MgCl₂ at 3.5×10⁶ cells per dish. The cells were seeded one day before and cultured overnight at 37° C. and 10% CO₂. At day 1, transfections were performed in 49 dishes at 37° C. with lipofectamine (Gibco) with 2 μg pCD46-3000/Neo per dish using procedures well known to persons skilled in the art. After 5 h, the medium of the cells was replaced by DMEM supplemented with 10% FCS and 1% MgCl₂. Replacement of the medium was performed regularly. The selection pressure was held at 500 μg G418 per liter. Of the colonies of cells that grew out, 571 were picked manually (on day 20, 21 and 22) to 96-well plates. After several weeks of growth in DMEM+serum, in the presence of G418, the culture supernatant from these clones was tested for the presence of monoclonal antibody by ELISA analysis using methodologies well known to persons skilled in the art (IgG1 light chain/heavy chain capture: using anti-human IgG1 kappa antibody MαH-Ig, Pharmingen cat.nr. 555789 for capture and biotin labeled anti-human MαH-Ig cat.nr. 555869 from Pharmingen for staining and anti-human IgG1 antibody H-IgG1, Sigma cat.nr. I-3889 for the control standard curve).

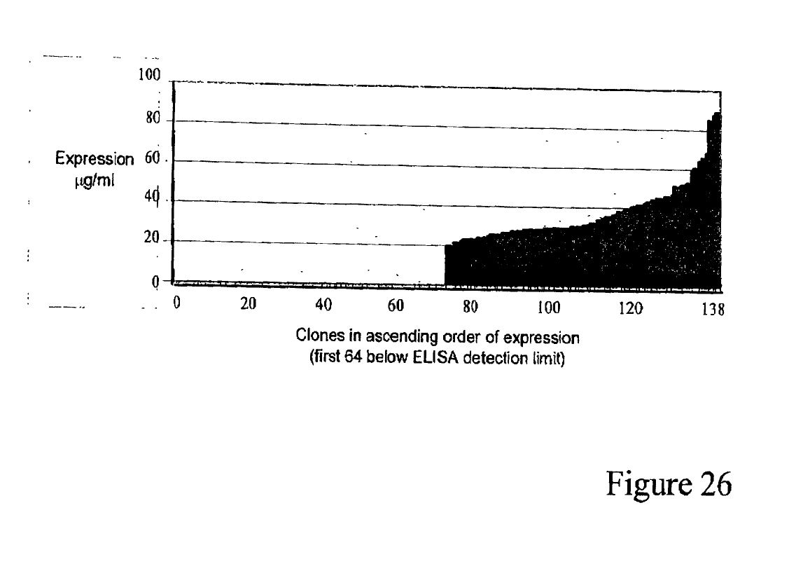

On the first ELISA, 124 (22%) of clones failed to express antibody (below the cut off of the ELISA), and the top expressing clones (138 clones) were above the upper detection limit of the ELISA test (more than 700 ng/ml). Therefore a second ELISA was performed on the 138 clones that produced the highest amount of antibody. The results of this second ELISA in ascending order of expression are shown in FIG. 26. Because all of the used samples were diluted to fit to the standard curve of the second ELISA it was found that 64 clones expressed levels that were below the detection level of the ELISA, after dilution. Batches of all these 138 clones, as well as 97 additional fast-growing clones, were frozen. A relatively small number of clones produced very high levels of antibody, and 37 of these were passed for stability tests and small-scale antibody production. The 37 clones expressing high levels of recombinant K53/IgG1 were subcultured and the antibody production was determined as pg per cell per day. For this 1×10⁶ cells per clone were seeded per well in a 6 well plate (2 wells) giving a total seed of 2×10⁶ cells and a sample was taken after 4 days of culturing in medium without selection. This procedure was performed twice and the measured production rate ranged between 0.13 and 21.99 pg/cell/day.

Stability tests were performed on the 37 selected clones. One sample was taken at time zero, another after 4 weeks of culturing, and these samples were tested for monoclonal production. One clone immediately lost production capability, there were technical problems with 3 clones, and only 1 clone exhibited a very marked reduction in expression level (i.e. showed instability). All other 32 clones gave reasonably stable production of monoclonal antibody. These clones were also passaged to serum-free JRH ExCell-525 medium and the supernatant collected for subsequent purification and analysis. These clones were taken through one round of limiting dilutions. For this, cells were counted using procedures known to the skilled artisan and seeded in 96 well plates (4 plates per clone) at a density of 0.3 cells per well. The limiting dilution efficiency was between 2-15%. Of each clone about 5-14 sub-clones were passed from 96 wells to 24 wells (selected on growth). An ELISA was performed, and most sub-clones were positive for antibody production. Two vials of all sub-clones were frozen. Based on criteria of expression levels, stability of expression and initial glyco-analysis results, five clones were subjected to a further round of limiting dilution to ensure that all were indeed single clones. These clones were named CD46-007, -114, -124, -130 and -233. Sub-clones of the five clones were used for further culturing and analysis of antibody production levels. Of these clones, CD46-124 was a relatively bad growing cell line and left out of further experimentation. The other sub-clones were tested in ELI SA and one clone of each was chosen for further experiments. The best performers reached production levels that ranged between 6.35 and 15.00 pg/cell/day.

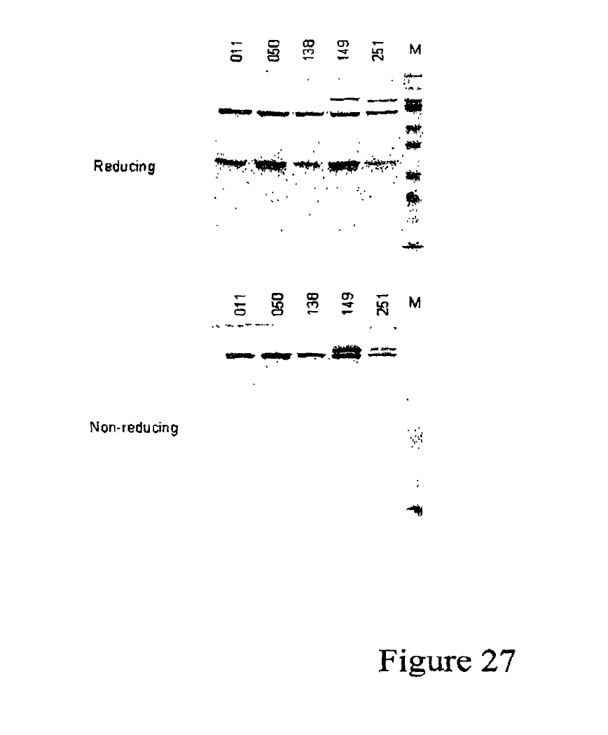

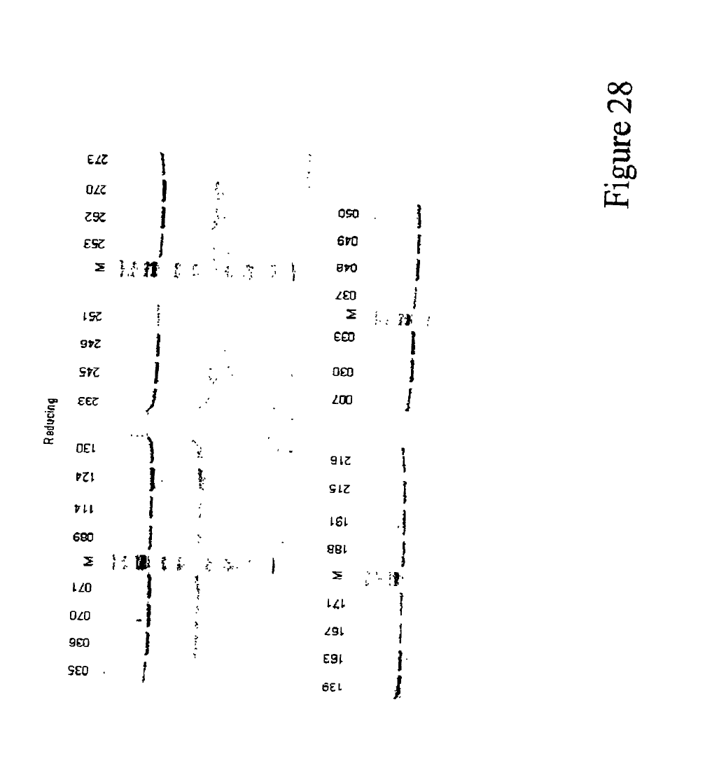

Culture supernatants from several PER.C6 clones producing recombinant K53/IgG1 were purified using protein-A columns (Econo-Pac colums prepacked with 2 ml of Affi-Gel protein-A agarose). The columns were rinsed with 50 ml PBS and subsequently the supernatant was applied on the column. After rinsing the column with 20 ml PBS, bound proteins were eluted with 0.1 M citric acid pH 3.0. Fractions of 1 ml were collected and immediately neutralized with 200 μl 1 M Tris. After elution the column was rinsed with at least 30 ml PBS before the next purification round was started. In total, three different protein-A columns were used to purify K53/IgG1 from all clones. The protein containing fractions, as determined by spectrophotometry at OD₂₈₀ nm, were pooled and dialyzed extensively against PBS at 4° C. Finally the products were filtered (0.22 μm) and stored at 4° C. The protein concentration of all samples was determined using the Biorad Protein Assay according to the micro-assay procedure. The integrity of the antibody after purification was determined by SDS-PAGE followed by Coomassie Brilliant Blue staining. For each clone, 25 μl of the purified antibody was separated on a 10% reducing or a 6% non-reducing SDS-PAGE gel using the Biorad Mini Protean 3 system using general procedures known to persons skilled in the art. Tested on SDS-PAGE under reducing conditions, most clones showed two protein bands of approximately 55 and 30 kDa, that most likely correspond with the IgG1 heavy and light chain respectively. No other protein bands were detected. However, two clones, number 149 and 251, showed an extra protein band of approximately 80 kDa. An extra band of approximately 200 kDa was visible when purified antibody of these two clones was separated under non-reducing conditions. FIG. 27 and 28 show the results of the SDS-PAGE and Coomassie Brilliant Blue staining.

For the expression of the recombinant K53/IgG1 in HEK 293 cells, the following procedures were conducted. Transfections were performed using Fugene 6™ transfection agent (Roche) according to the manufacturer recommendations. For the transfection, HEK 293 cells were grown at approximately 50-60% confluency in a six well plate in 2 ml/well DMEM supplemented with 10% FCS and penicillin 10000 IU/ml and streptomycin 10000 μg/ml (pen/strep). The cells were seeded the day before and cultured overnight at 37° C. and 5% CO₂. Co-transfections (day 1) were performed in 10 cm culture-dish plate at 37° C. and 5% CO₂. Cells were refreshed with 6 ml/dish fresh DMEM with 10% FCS and pen/strep. 15 μl Fugene 6™ agent was carefully pipetted into 600 μl of DMEM and incubated for 5 min at room temperature. Subsequently, 3 μg pcDNA3.1 K53/Zeo (containing the K53/IgG1 heavy chain) and 3 μg pcDNA3.1 κ-K53/Zeo (containing the kappa 2 light chain under control of the CMV promoter) was added to the DMEM/Fugene mixture and incubated for 15 min at room temperature. These procedures were therefore different from the procedures used for the PER.C6 cells, since here two separate plasmids were transfected, while pCD46-3000/Neo used for PER.C6 cells was a single plasmid encoding both heavy and light chain. 600 μl per dish of the obtained transfection mixture was distributed by pipetting small droplets. At day 2, cells were refreshed with 6 ml fresh DMEM with 10% FCS, and pen/strep. At day 3, the cells were refreshed with cold DMEM with 10% FCS, pen/strep, supplemented with Zeocin (Invitrogen) 500 μg/ml. Cells were left at 4° C. for 3.5 h, and thereafter transferred into the incubator at 37° C. and 5% CO₂. Cells that did not integrate at least one of the two plasmids containing the Zeocin resistance gene, are considered to be killed in the selection procedure. Every 2 to 4 days, the cells were refreshed with medium containing the same concentration of zeocin. After 3 weeks, 13 distinct colonies were picked by scraping and pipetting the cells from the bottom of the dish. Cells were transferred into a 6-wells plate containing 2 ml medium with zeocin. Colonies were expanded for 2 weeks before they were tested for antibody production using an IgG-specific ELISA. The 5 highest producing colonies named L53-1, -2, -7, -8 and -10 were selected for further growth and frozen in liquid N₂.

Of the five selected clones, clone L53-7 was selected as best candidate for antibody production. Clone L53-2 was not stable and turned out to have lost antibody production over time. Furthermore, clones L53-1 and -8 could not be subcultured. L53-7 was expanded in selection medium in triple flasks, and grown until a confluency of approximately 80% was reached. Ultra-CHO medium (100-120 ml) per flask was added and cells were further cultured for 3 days. Supernatant containing the antibody was harvested, cells and debris were spun down and supernatant was filtered (0.22 μm). The antibodies were purified using a protein A-sepharose column. The column was rinsed with 50 ml PBS, and subsequently supernatant was run over the column. After rinsing the column with at least 20 ml PBS, the proteins were eluted with 0.1 M citric acid pH 3.0. Fractions of 1 ml were collected and immediately neutralized with approximately 200 μl 1 M Tris. The purification was performed 5 times (H1-5). The protein containing fractions 3 and 4 (H1, 2, 3 and 4), as determined by spectrophotometry at 280 nm, were pooled and dialyzed extensively against PBS at 4° C. The monoclonal antibodies were filtered (0.22 μm) and final concentrations were determined using the Biorad Protein Assay (see, Table XI).

### Example 15

Neutralization of CD46 Augments Killing of Tumor Cells Targeted With Anti-EpCAM Antibody UBS-54

To detect the effect of neutralizing complement regulatory proteins like CD46, the following experiments are performed. A 1×10⁶ CD46-overexpressing tumor cells (LS174T) are injected subcutaneously in immune deficient mice and treated one day later with 100 μg human anti-EpCAM (UBS-54) monoclonal antibody and/or 100 μg human K53/IgG1 monoclonal antibody. On day 3 and 6, the treatment is repeated with 50 μg UBS-54 monoclonal antibody and/or 50 μg human K53/IgG1 monoclonal antibody. As a control a Streptococcus specific GBS III antibody is used (see above). The effect of monoclonal antibody treatment is evaluated by measuring the mean tumor size (maximal length×maximal height×maximal width) during 3 weeks (see above). This shows that K53/IgG1 is also useful in the context of other anti-tumor therapeutics.

References

Alexanian R and Dimopoulos M (1994) The treatment of multiple myeloma. N Engl J Med 7:484

Attal M, Harousseau J L, Stoppa A M, et al. (1996) A prospective, randomized trial of autologous bone marrow transplantation and chemotherapy in multiple myeloma. N Engl J Med 335:91

Baselga J, Tripathy D, Mendelsohn J, et al. (1996) Phase II study of weekly intravenous recombinant humanized anti-p185HER2 monoclonal antibody in patients with HER2/neu overexpressing metastatic breast cancer. J Clin Oncol 14:737

Bjorkstrand B, Ljungman P, Bird J M, et al. (1995) Autologous stem cell transplantation in multiple myeloma: results of the European Group for Bone Marrow Transplantation. Stem Cells 13:140S

Boccadoro M A, Alexanian R, Barlogie B (1997) Diagnosis, prognosis and standard treatment of multiple myeloma. Clin Hematol Oncol North Am 11:111

Bodey B, Bodey Jr B and Siegel SE (2000) Genetically-engineered monoclonal antibodies for direct anti-neoplastic treatment and cancer cell-specific delivery of chemotherapeutic agents. Curr Pharm Des 27:49

Boel E, Verlaan S, Poppelier M J J G, et al. (2000) Functional human monoclonal antibodies of all isotypes constructed from phage display library-derived single chain Fv antibody fragments. J Immunol Methods 239:153

Bruggeman M and Neuberger M S (1996) Strategies for expressing human antibody repertoires in transgenic mice. Immunol Today 17:391

Burton D R and Barbas III C F (1994) Human antibodies from combinatorial libraries. Adv Immunol 57:191

Cunningham D, Paz-Ares L, Milan S, et al. (1994) High-dose melphalan and autologous bone marrow transplantation as consolidation in previously untreated myeloma. J Clin Oncol 12:759

Cumow R T (1997) Clinical experience with CD64-directed immunotherapy. An overview. Cancer Immunol Immunother 45:210

De Kruif J, Terstappen L, Boel E et al. (1995 a) Rapid selection of cell subpopulation-specific human monoclonal antibodies from a synthetic phage antibody library. Proc Natl Acad Sci USA 92:3938

De Kruif J, Boel E, Logtenberg T (1995b) Selection and application of human single chain Fv antibody fragments from a semi-synthetic phage antibody display library with designed CDR3 regions. J Mol Biol 248:97

De Kruif J, Van der Vuurst de Vries A-R, Cilenti L, et al. (1996) New perspectives on recombinant human antibodies. Immunol Today 17:453

Dennis J W, Granowsky M and Warren C E (1999) Protein glycosylation in development and disease. BioEssays 21:412

Dorig R E, Marcil A, Chopra A, et al. (1993) The human CD46 molecule is a receptor for measles virus (Edmonston strain). Cell 75:295

Elliot M J, Maini R N, Feldman M, et al. (1994) Repeated therapy with monoclonal antibody to tumor necrosis factor alfa (cA2) in patients with rheumatoid arhritis. Lancet 344:1125

Engelmann S, Ebeling O, Schwartz-Albiez R (1995) Modulated glycosylation of proteoglycans during differentiation of human B lymphocytes. Biochim Biophys Acta 1267:6

Farooq M, Takahashi N, Arrol H, et al. (1997) Glycosylation of polyclonal and paraprotein IgG in multiple myeloma. Glycoconj J 14:489

Foote J and Winter G (1992) Antibody framework residues affecting the the conformation of the hypervariable loops. J Mol Biol 224:487

Granziero L, Nelboeck P, Bedoucha M, et al. (1997) Baculovirus cDNA libraries for expression cloning of genes encoding cell-surface antigens. J Immunol Methods 203:131

Hakimi J, Chizzonite R, Luke D R, et al. (1991) Reduced immunogenicity and improved pharmacokinetics of humanized anti-Tac in cynomolgus monkeys. J Immunol 147:1352

Hara T, Kuriyama S, Kiyohara H, et al. (1992) Soluble forms of membrane cofactor protein (CD46, MCP) are present in plasma, tears, and seminal fluid in normal subjects. Clin Exp Immunol 89:490

Harousseau J-L, Attal M, Divine M, et al. (1995) Autologous stem cell transplantation after first remission induction treatment in multiple myeloma: a report of the French Registry on Autologous Transplantation in Multiple Myeloma Blood 85:3077

Havenga M J, Werner A B, Valerio D, et al. (1998) Methotrexate selectable retroviral vectors for Gaucher disease. Gene Ther 5:1379

Huls G A., Heijnen I A F M., Cuomo M E., et al. (1999) A recombinant, fully human monoclonal antibody with antitumor activity constructed from phage-displayed antibody fragments. Nature Biotechnol 17:276

Iwata K, Seya T, Yanagi Y, et al. (1995) Diversity of sites for measles virus binding and for inactivation of complement C3b and C4b on membrane cofactor protein CD46. J Biol Chem 270:15148

Jones P T, Dear P H, Foote J, et al. (1986) Replacing the complementarity-determining regions in a human antibody with those from a mouse. Nature 321:522

Juhl H, Helmig F, Baltzer K, et al. (1997) Frequent expression of complement resistance factors CD46, CD55 and CD59 on gastrointestinal cancer cells limits the theurapeutical potential of monoclonal antibody 17-1A. J Surg Oncol 64:222

Jurianz K, Maslak S, Garcia-Schuler H, et al. (1999) Neutralization of complement regulatory proteins augments lysis of breast carcinoma cells targeted with rhumAb anti-HER2. Immunopharmacology 42:209

Khazaeli M B, Saleh M N, Liu T P, et al. (1989) Pharmacokinetics and immune response of 131I-chi-meric mouse/human B72.3 (human gamma4) monoclonal antibody in humans. Cancer Res 51:5461

King C A, Spellerberg M B, Zhu D, et al. (1998) DNA vaccines with single-chain Fv fused to fragment C. of tetanus toxin induce protective immunity against lymphoma and myeloma. Nature Med 4:1281

Kinugasa N, Higashi T, Nouso K, et al. (I 999) Expression of membrane cofactor protein (MCP, CD46) in human liver diseases. Br J Cancer 80:1820

Köhler G and Milstein C. (1975) Continuous cultures of fused cells secreting antibody of predefined specificity. Nature 256:495

Liszewski M K, Post T W, Atkinson J P (1991) Membrane cofactor protein (MCP or CD46): newest member of the regulators of complement activation gene cluster Annu Rev Immunol 9:431

Liszewski M K, Leung M K, Atkinson J P (I 998) Membrane cofactor protein: importance of N- and O-glycosylation for complement regulatory function. J Immunol 161:3711

Maloney D G and Press O W (1998) Newer treatments for non-Hodgkin's lymphoma: monocloanl antibodies. Oncology 12:63

Matsuda F and Honjo T (1996) Organization of the human immunoglobulin heavy-chain locus. Adv Immunol 62:1

McLaughlin P, Grillo-Lopez A J, Link B K, et al. (1998) Rituximab chimeric anti-CD20 monoclonal antibody therapy for relapsed indolent lymphoma: half of patients respond to a four-dose treatment program. J Clin Oncol 16:2825

Mendez M J, Green L L, Corvalan J R F, et al. (1997) Functional transplant of megabase human immunoglobulin loci recapitulates human antibody response in mice. Nature Genetics 15:146

Miller R A, Oseroff A R, Stratte P T et al. (1983) Monoclonal antibody therapeutic trials in seven patients with T-cell lymphoma. Blood 62:988

Murray K P, Mathure S, Kaul R, et al. (2000) Expression of complement regulatory proteins-CD35, CD46, CD55 and CD59- in benign and malignant endometrial tissue. Gynecol Oncol 76:176

Naniche D, Varior-Krishnan G, Cervoni F, et al. (1993) Human membrane cofactor protein (CD46) acts as a cellular receptor for measles virus. J Virol 67:6025

Oglesby T J, Allen C J, Liszewski M K, et al. (1992) Membrane cofactor protein (CD46) protects cells from complement mediated attack by an intrinsic mechanism. J Exp Med 175:1547

Post T W, Liszewski M K, Adams E M, et al. (1991) Membrane cofactor protein of the complement system: alternative splicing of serine/threonine/proline-rich exons and cytoplasmic tails produces multiple isoforms that correlate with protein phenotype. J Exp Med 174:93

Renner C. and Pfreundschuh M (1995) Tumor therapy by immune recruitement with bispecifc antibodies. Immunol Rev. 145:179

Riechmann L, Clark M, Waldmann H et al. (1988) Reshaping human antibodies for therapy. Nature 332:323

Riethmuller G, Holz E, Schlimok G, et al. (1998) Monoclonal antibody therapy for resected Dukes'C colorectal cancer: seven-year outcome of a multicenter randomized trial. J Clin Oncol 16:1788

Schmitt C A, Schwaeble W, Wittig B M, et al (1999) Expression and regulation by interferon-gamma of the membrane-bound complement regulators CD46 (MCP), CD55 (DAF) and CD59 (protectin) in gastrointestinal tumors. Eur J Cancer 35:117

Schneider-Gadicke E and Riethmuller G (1995) Prevention of manifest metastasis with monoclonal antibodies: A novel approach to immunotherapy of solid tumors. Eur J Cancer 31A:1326

Seya T, Hara T, Matsumoto M, et al. (1990a) Quantitative analysis of membrane cofactor protein (MCP) of complement. High expression of MCP on human leukemia cell lines, which is down-regulated during cell differentiation. J Immunol 145:238

Seya T, Hara T, Matsumoto M, et al. (1990b) Complement mediated tumor cell damage induced by antibodies against membrane cofactor protein (MCP, CD46). J Exp Med 172:1673

Shawler D L, Bartholomew R M, Smith L M et al. (1985) Human immune response to multiple injections of murine monoclonal IgG. J Immunol 135:1530

Simpson K L, Jones A, Norman S, et al. (1997) Expression of the complement regulatory proteins decay accelerating factor (DAF, CD55), membrane cofactor protein (MCP, CD46) and CD59 in the normal human uterine cervix and in premalignant and malignant cervical disease. Am J Pathol 151:1455

Slupsky J R, Duggan-Keen M, Booth L A, et al. (1993) The peanut-agglutinin (PNA)-binding surface components of malignant plasma cells. Br J Haematol 83:567

Stephens S, Emtage S, Vetterlei O, et al. (1995) Comprehensive pharmacokinetics of a humanized antibody and analysis of residual anti-idiotypic responses. Immunology 85:668

Terstappen L W, Johnsen S, Segers-Nolten I M, et al. (1990) Identification and characterization of plasma cells in normal human bone marrow by high-resolution flow cytometry Blood 76:1739.

Thorsteinsson L, O'Dowd G M, Harrington P M, et al. (1998) The complement regulatory proteins CD46 and CD59, but not CD55, are highly expressed by glandular epithelium of human breast and colorectal tumor tissues. APMIS 106:869

Tricot G, Jagannath S, Vesole D, et al. (1995) Peripheral blood stem cell transplant for multiple myeloma: identification of favourable variables for rapid engraftment in 225 patients. Blood 85:588

Urlaub G, Kas E, Carothers A M, et al. (1983) Deletion of the diploid dihydrofolate reductase locus from cultured mammalian cells. Cell 33:405

Van der Vuurst de Vries A-R and Logtenberg T (2000) Dissecting the human peripheral B cell compartment with phage display derived antibodies. Immnunology (in press)

Van Dixhoorn M G, Dekker S, Janssen R W, et al. 00) Human CD46 rather than CD55 is a key element in protection against complement activation in vitro. Transplant Proc 32:916

Vaughan T J, Osbourn J K and Tempest P R (1998) Human antibodies by design. Nat Biotechnol 16:535

Vile R G and Chong H (1996) Immunotherapy III: Combinatorial molecular immunotherapy: a synthesis and suggestions—Cancer Metast Rev 15:351

What is claimed is:

1. A process for identifying a disease associated molecular marker which disease associated molecular marker is associated with a subset of cells, said process comprising the steps of:
a) incubating cells of a species with a library of binding molecules, combined with an incubation with diseased cells of the species;
b) obtaining, from said incubation, a collection of diseased cells essentially free from non-diseased cells, by sorting said collection of diseased cells from non-diseased cells according to parameters that distinguish between said collection of diseased cells and said non-diseased cells;
c) obtaining binding molecules from said collection of diseased cells;
d) selecting, from said obtained binding molecules, an individual binding molecule that preferentially binds to said diseased cells in comparison to binding to said non-diseased cells;
e) identifying a molecular marker which, in its disease associated form, binds to said individual binding molecule selected under step d), said disease associated molecular marker being associated with said collection of diseased cells obtainable according to step b); and
f) establishing that said disease associated molecular marker has a counterpart associated with non-diseased cells wherein said counterpart is less capable of binding said individual binding molecule.

2. The process according to claim 1, further comprising the step of:
establishing that said counterpart and said disease associated molecular marker differ in at least one post-translational modification.

3. The process according to claim 2, wherein said post-translational modification is a glycosylation modification.

4. The process according to any one of claims 1-3, further comprising the steps of:
recovering said individual binding molecule which binds to said disease associated molecular marker; and
characterizing said individual binding molecule.

5. A process for identifying a binding molecule capable of binding a subset of diseased cells, said process comprising the steps of:
a) incubating cells of a species with a library of binding molecules, combined with an incubation with diseased cells of said species;
b) obtaining, from said incubation, a collection of diseased cells essentially free of non-diseased cells, by sorting said collection of diseased cells from non-diseased cells according to parameters which distinguish between said collection of diseased cells and said non-diseased cells;
c) obtaining binding molecules from said collection of diseased cells;
d) selecting, from said obtained binding molecules, an individual binding molecule capable of preferentially binding to said diseased cells as compared to binding to said non-diseased cells;
e) recovering said individual binding molecule selected in step d); and
f) establishing that said individual binding molecule preferentially binds to disease associated molecular marker, said disease associated molecular marker being associated with said diseased cells, said disease associated molecular marker further having a counterpart associated with non-diseased cells wherein said counterpart is less capable of binding said individual binding molecule.

6. The process according to claim 5, further comprising the step of:
establishing that said counterpart and said disease associated form differ in at least one post-translational modification.

7. The process according to claim 6, wherein said post-translational modification is a glycosylation modification.

8. The process according to any one of claims 1-7, wherein said sorting is performed using a molecule that preferentially interacts with said diseased cells as compared to said non-diseased cells.

9. The process according to any one of claims 1-8, wherein said sorting is performed with a molecule that preferentially interacts with said non-diseased cells as compared to said diseased cells.

10. The process according to any one of claims 1-9, wherein said library of binding molecules is a phage antibody display library.

11. The process according to claim 10, wherein said phage display library comprises at least 1 ×10⁸ specificities.

12. The process according to any one of claims 1-11, wherein said sorting is conducted using a fluorescence activated cell sorter.

13. The process according to any one of claims 1-12, wherein said parameters are fluorescence based parameters.

14. The process according to any one of claims 1-13, wherein said diseased cells are present in a cell population derived from a mammalian species suffering from cancer, diabetes, Alzheimer's disease, multiple sclerosis, rheumatoid arthritis, inflammatory disease or viral infections.

15. The process according to any one of claims 1-14, wherein said diseased cells are tumor cells.

16. The process according to claim 15, wherein said tumor cells are selected from the group consisting of multiple myeloma cells, breast tumor cells and colon carcinoma cells.

17. A disease associated molecular marker produced by the process according to any one of claims 1-16.

18. A binding molecule produced by a process according to any one of claims 1-16.

19. The binding molecule of claim 18, wherein said disease associated molecular marker is a CD46 protein.

20. The binding molecule of claim 19, wherein said CD46 protein comprises human CD46 protein.

21. A binding molecule capable of specifically binding to an epitope present in a subset of CD46 proteins.

22. The binding molecule of claim 21, said binding molecule capable of distinguishing a subset of CD46 comprising cells.

23. The binding molecule of claim 22, wherein said subset of CD46 comprising cells comprises a hemopoietic cell, a cervix cell, a colon cell, a kidney cell or a liver cell.

24. The binding molecule of claim 23, wherein said hemopoietic cell is derived from a B-cell.

25. The binding molecule of any one of claims 18-24, capable of binding to a multiple myeloma cell.

26. The binding molecule of any one of claims 21-25, wherein said CD46 protein comprises human CD46 protein.

27. The binding molecule of any one of claims 18-26, wherein said binding molecule is an antibody or part or derivative thereof having the binding activity of an antibody.

28. The binding molecule of claim 27, wherein said binding molecule is a human or humanized antibody.

29. The binding molecule of any one of claims 18-28, further comprising a tag associated with said binding molecule.

30. The binding molecule of claim 29, wherein said tag comprises a toxin, a radioactive substance, or a toxin and a radioactive substance.

31. A method for treating a subject suffering from, or at risk of suffering from, a disease, said method comprising:
administering to said subject a therapeutically acceptable amount of the binding molecule of any one of claims 18-30.

32. The method according to claim 31, wherein said disease is a neoplastic disease.

33. Use of a binding molecule according to any one of claims 18-30 for the preparation of a medicament.

34. Use according to claim 33, for the treatment of a neoplastic disease.

35. A method for typing a cell, said method comprising:
determining whether the cell specifically binds the binding molecule of any one of claims 18-30.

36. Use of an epitope expressed on a subset of CD46 expressing cells as a marker for neoplastic cells.

37. A nucleic acid encoding a binding molecule, or a part thereof, according to any one of claims 18-30.

38. A cell comprising a nucleic acid according to claim 37.

39. The cell of claim 38, wherein said cell is a primate, rodent, bird, or plant cell.

40. The cell of claim 38 or claim 39, wherein said cell is a human cell.

41. The cell of any one of claims 38-40, said cell further comprising:
means for the conditional expression of a nucleic acid of interest.

42. The cell of claim 41, wherein said means for the conditional expression of a nucleic acid of interest comprises a tetracycline responsive expression system.

43. The cell of any one of claims 38-42, wherein said cell comprises a nucleic acid encoding an early protein of adenovirus or a functional part, derivative and/or analogue thereof.

44. The cell of claim 43, wherein said early protein comprises adenovirus E1 or a functional part, derivative and/or analogue thereof.

45. The cell of claim 43 or claim 44, said cell further comprising:
adenovirus E2A or a functional part, derivative and/or analogue thereof.

46. A plant or a non-human animal comprising the cell of any one of claims 38-45.

47. The plant or non-human animal of claim 46, said plant or non-human animal being transgenic for the nucleic acid of claim 37.

48. A gene delivery vehicle comprising the nucleic acid of claim 37.

49. A kit comprising the binding molecule of any one of claims 18-30.
